SEQUENCE LISTING (5 sequences)

SEQ ID NO: 1 - DNA, 810 bp, ARTIFICIAL SEQUENCE
  Description of Artificial Sequence ssDNA Oligonucleotide
atgactaagcaaccaccaatcgcaaaagccgatctgcaaaaaactcagggaaaccgtgcaccagcagcagttaaaaatagcgacgtgattagttttattaaccagccatcaatgaaagagcaactggcagcagctcttccacgccatatgacggctgaacgtatgatccgtatcgccaccacagaaattcgtaaagttccggcgttaggaaactgtgacactatgagttttgtcagtgcgatcgtacagtgttcacagctcggacttgagccaggtagcgccctcggtcatgcatatttactgccttttggtaataaaaacgaaaagagcggtaaaaagaacgttcagctaatcattggctatcgcggcatgattgatctggctcgccgttctggtcaaatcgccagcctgtcagcccgtgttgtccgtgaaggtgacgagtttagcttcgaatttggccttgatgaaaagttaatacaccgcccgggagaaaacgaagatgccccggttacccacgtctatgctgtcgcaagactgaaagacggaggtactcagtttgaagttatgacgcgcaaacagattgagctggtgcgcagcctgagtaaagctggtaataacgggccgtgggtaactcactgggaagaaatggcaaagaaaacggctattcgtcgcctgttcaaatatttgcccgtatcaattgagatccagcgtgcagtatcaatggatgaaaaggaaccactgacaatcgatcctgcagattcctctgtattaaccggggaatacagtgtaatcgataattcagaggaataa

SEQ ID NO: 2 - DNA, 786 bp, ARTIFICIAL SEQUENCE
  Description of Artificial Sequence ssDNA Oligonucleotide
atgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatga

SEQ ID NO: 3 - DNA, 3997 bp, ARTIFICIAL SEQUENCE
  Description of Artificial Sequence ssDNA Oligonucleotide
ctgcggccgcaaggcaattgtggagtcgaggaattctaccgggtaggggaggcgcttttcccaaggcagtctggagcatgcgctttagcagccccgctgggcacttggcgctacacaagtggcctctggctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccggtggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgccgtttccctgccacagtctgagagctccctggcgaattcggtaccaataaaagagctttattttcatgatctgtgtgttggtttttgtgtgcggcgcgccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 4 - DNA, 220 bp, ARTIFICIAL SEQUENCE
  Description of Artificial Sequence ssDNA Oligonucleotide
cttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggcta

SEQ ID NO: 5 - DNA, 220 bp, ARTIFICIAL SEQUENCE
  Description of Artificial Sequence ssDNA Oligonucleotide
tagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaag

(19) United States
(12) Patent Application Publication
(10) Pub. No.: US 2004/0101520 A1
(43) Pub. Date: May 27, 2004

(54) Recombination method

(76) Inventors: Joep P P Muijrers, Dresden (DE); Youming Zhang, Dresden (DE); Andrian Francis Stewart, Dresden (DE)

Correspondence Address: WENDEROTH, LIND & PONACK, L.L.P., 2033 K STREET N. W., SUITE 800, WASHINGTON, DC, 20006-1021, US

(21) Appl. No.: 10/470,679

(22) PCT Filed: Feb. 8, 2002
(86) PCT No.: PCT/IB02/01415

(30) Foreign Application Priority Data
Feb. 9, 2001 (GB) 0103276.2
Aug. 21, 2001 (GB) 0120312.4

(51) Int. Cl. A61K 48/00; C12N 15/85; C12N 15/74

(52) U.S. Cl. 424/93.21; 435/455; 435/489

(57) ABSTRACT

The invention relates to a novel method for altering the sequence of a nucleic acid molecule using repair recombination in a simple one component system. The frequency of the recombination reaction is high, allowing a range of feasible selection strategies to identify successful recombination events. The method involves the steps of bringing a first nucleic acid molecule into contact with a second nucleic acid molecule in the presence of a phage annealing protein into contact with a second nucleic acid molecule in the presence of a phage annealing protein, or a functional equivalent or fragment thereof, wherein said first nucleic acid molecule comprises at least two regions of shared sequence homology with the second nucleic acid molecule, under conditions suitable for repair recombination to occur between said first and second nucleic acid molecules; ad selecting a nucleic acid molecule whose sequence has been altered so as to include sequence from said second nucleic acid molecule.

The invention relates to a novel method for altering the sequence of a nucleic acid molecule using repair recombination in a simple one component system. The frequency of the recombination reaction is high, allowing a range of feasible selection strategies to identify successful recombination events.

All publications, patents and patent applications cited herein are incorporated in full by reference.

The engineering of nucleic acid molecules, particularly DNA molecules, is of fundamental importance to Life Science research. For example, the construction and precise manipulation of nucleic acid molecules is required in many studies and applications in the research fields of, for example, functional genomics (for review, see Vukmirovic and Tilghman, Nature 405 (2000), 820-822), structural genomics (for review, see Skolnick et al., Nature Biotech 18 (2000), 283-287) and proteomics (for review, see Banks et al., Lancet 356 (2000), 1749-1756; Pandey and Mann, Nature 405 (2000), 837-846).

A number of methods are currently available for engineering nucleic acid molecules, particularly DNA molecules. Conventional methods, which are still the most widely used, rely on restriction digestion, followed by ligation (see Sambrook J and Russell D. W. Molecular Cloning, a laboratory manual, 3^{rd} ed. (2000) Cold Spring Harbor Laboratory Press, Cold Spring Harbor, N.Y.). Progress in-our understanding of the various mechanisms of nucleic acid recombination has allowed conventional cloning techniques to be complemented and partially replaced by more advanced strategies utilising homologous recombination (in prokaryotes, see below; in eukaryotes, see, for example, Bode et al., Biol Chem. 381 (2000), 801-813; Joyner, Gene Targeting, a practical approach, (2000) second edition, Oxford University Press Inc. New York), PCR-directed mutagenesis (see Ling and Robinson, Anal. Biochem. 254 (1997), 157-178), site-specific recombination (for example, Hauser et al., Cells Tissues Organs 167 (2000), 75-80) and transposon mutagenesis (see, for example, Martienssen, Proc. Natl. Acad, Sci. USA 95 (1998), 2021-2026; Parinov and Sundaresan, Curr Opin Biotechnol 11 (2000), 157-161).

However, these techniques often contain inherent complications. For example, although PCR-based in vitro strategies allow precise site-directed mutagenesis to be effected, such methods suffer from the introduction of unwanted artefactual secondary mutations in the targeted molecule during amplification of the mutated nucleic acid product. Furthermore, this method is presently limited to molecules of a maximal size of around 10-15 kilobasepairs. Other techniques do not allow flexible DNA engineering at any chosen position, but instead require specific sequence elements (site-specific recombination based methods) or are used for random targeting (transposon based methods). Also, long homology lengths are currently required for DNA engineering in eukaryotes by homologous recombination.

The application of homologous recombination in DNA engineering has been pioneered in S. cerevisiae (for review see Shashikant et al., Gene 223 (1998), 9-20). However, since several inherent complications limit the usefulness of yeast as a DNA engineering host, homologous recombination based DNA engineering has recently been established in the premier cloning host, E. coli. (for review see Muyrers et al., Trends in Bioch Sci, (2001) 26(5): 325-331). To date, three major recombination pathways have been described in Escherichia coli. All of these pathways have in some way or another been used for recombinogenic DNA engineering (for review, see Muyrers et al., Trends in Bioch Sci, (2001) 26(5): 325-331).

The most widely conserved pathway is the RecA-dependent recombination pathway, which is responsible for the majority of recombinogenic processes in the bacterial cell. In many such recombinogenic processes, RecA, the most prominent strand invasion protein in evolution, functionally cooperates with RecBCD, a large holoenzyme composed of RecB, RecC and RecD subunits, which amongst other functions exhibits vigorous exonuclease activity (for review see Kowalczykowski et al., Microbiol Rev 58 (1994), 401-465; Kuzminov, Microbiol Mol Biol Rev 63 (1999), 751-813).

A second recombination pathway is the RecF-pathway. This pathway depends on interactions between a large group of proteins, including RecF and, most likely, RecA, and is activated by the sbcBCD mutation (Ryder et al., Genetics 143 (1996), 1101-1114; Phillips et al., J Bacteriol 170 (1998), 2089-2094; Cromie et al., Genetics 154 (2000), 513-522).

In the third pathway, recombination requires the expression of both components of the RecE/RecT protein pair, or of its functional homologues derived from the lambda phage, Redα/Redβ. The functional homology of these protein pairs is evident from the findings that both RecE and Redα are 5′ to 3′ exonucleases, and both RecT and Redβ are annealing proteins that display various similar activities in vitro and in vivo. A variety of studies have concluded that RecE/RecT and Redα/Redβ are functionally equivalent (Hall and Kolodner, Proc. Natl. Acad. Sci. USA 91 (1994), 3205-3209; Kolodner et al., Mol Microbiol 11 (1994), 23-30; Muyrers et al., Genes Dev 14 (2000), 1971-1982).

In the last few years, a technology termed ET recombination has been developed that uses the RecE/RecT protein pair (or its functionally homologous pair Redα/Redβ) for precise DNA engineering (Zhang et al., Nature Genet 20 (1998), 123-128; Muyrers et al., Nucl Acids Res 27 (1999), 1555-1557; co-owned, co-pending International patent application WO99/29837; for review see Muyrers et al., Trends Bioch Sci (2001) 26(5): 325-331). ET recombination is widely applicable to a range of DNA modifications. Furthermore, this method can be used to clone DNA sequences from complex mixtures such as genomic DNA and Bacterial Artificial Chromosomes (BACs) in a single step, thereby providing a high-fidelity alternative to PCR amplification (Zhang et al., Nature Biotech 18 (2000), 1314-1317; also, co-owned, co-pending International patent application WO01/04288).

ET recombination functions through a RecA-independent recombination mechanism, which uses a specific functional cooperation (most likely through physical interaction) between both components of an orthologous protein pair (thus, a functional interaction is required between RecE and RecT, or between Redα and Redβ; Muyrers et al., Genes Dev 14 (2000), 1971-1982). Furthermore, each orthologous protein pair can mediate the required recombination reaction through two distinct recombination pathways, which are likely to be based on strand invasion and strand annealing, respectively (Muyrers et al., Genes Dev 14 (2000), 1971-1982).

This system is immensely powerful and may be used to introduce substitutions, deletions and insertions into nucleic acid molecules, as desired. However, the method is complicated by the need for expression, combined with regulation in some cases, of two proteins. In certain applications, achieving controlled expression of a component is therefore challenging. Also, ET recombination requires the absence or deactivation of the RecBCD holoenzyme. Also, the recombinogenic capacity needed for ET recombination can result in the appearance of unwanted internal deletions or rearrangements in the target nucleic acid molecule, especially if the target nucleic acid molecule contains significant repeats.

There thus remains a need for the development of a simple, efficient method for engineering nucleic acid molecules, that does not suffer from the problems mentioned above.

According to the invention, there is provided a method for altering the sequence of a nucleic acid molecule, said method comprising the steps of:

a) bringing a first nucleic acid molecule into contact with a second nucleic acid molecule in the presence of a phage annealing protein, or a functional equivalent or fragment thereof, wherein said first nucleic acid molecule comprises at least two regions of shared sequence homology with the second nucleic acid molecule, under conditions suitable for repair recombination to occur between said first and second nucleic acid molecules; and

b) selecting a nucleic acid molecule whose sequence has been altered so as to include sequence from said second nucleic acid molecule.

According to the invention, it has been discovered that phage annealing proteins such as RecT (from the rac prophage), Redβ (from phage λ), and Erf (from phage p22) display an activity in promoting repair recombination events, which activity is independent of any other phage-derived partner. In vitro, the formation of joint molecules between said first nucleic acid molecule and said second nucleic acid molecule is dependent only on the presence of the annealing protein. In vivo, no other exogenous components are required for the reaction, and no specific cellular manipulation is necessary for the method to proceed. For example, recBCD need not be inactivated; the method still works effectively in a recBCD+ background. The method is thus advantageous over methods previously described. In particular, the method is advantageous over ET recombination (Zhang et al., (1998); Muyrers et al., (1999); WO99/29837) in that it is not necessary for both the RecE and RecT proteins to be present. To distinguish the method further over ET recombination, the method may include the proviso that the RecE/Redα protein is not present during any sequence alteration reaction that is carried out in a prokaryotic cell. The method may be carried out in the presence of a single species of phage annealing protein, functional equivalent or fragment, although, as the skilled worker will appreciate, the presence of other, non-participating phage annealing proteins has no adverse effect on the method described herein. The method relies on a recombination event that involves the replacement of a section of replacement nucleic acid (the first nucleic acid molecule) for an equivalent section of target nucleic acid (the second nucleic acid molecule), to which it is directed through the existence of shared regions of sequence homology between the two molecule types. As with conventional homologous recombination events, the replacement nucleic acid becomes covalently attached to the target nucleic acid.

In this manner, the sequence information in the first nucleic acid molecule (the replacement nucleic acid molecule) becomes integrated into the second nucleic acid molecule (the target nucleic acid molecule) in a precise and specific manner, and with a high degree of fidelity.

The efficiency of the method is high, and allows the manipulation of sequences in a single step, without the need to apply any pressure using selectable genes. Furthermore, the regions of homology that are required between replacement and target nucleic acid are short, meaning that it is simple to generate molecules containing the nucleic acid sequence that is to be introduced, for example, by preparing or purchasing an oligonucleotide with the required sequence.

This method may be used for a number of different applications, such as, for example, precise site-directed mutagenesis, including deletion of sequences, insertion and substitution. The amount of sequence to be deleted, inserted or substituted may vary between one nucleotide (as in the introduction of point mutations) and nucleic acid molecules of many kilobasepairs in length. Examples of nucleic acid molecule types that can be suitably engineered include plasmids, such as targeting constructs used, for example, for ES cell targeting, Bacterial Artificial Chromosomes (BACs) used, for example, in transgenesis, and endogenous prokaryotic and eukaryotic chromosome(s).

Several differences between ET recombination and repair recombination have been found to exist, which clearly discriminate the recombination pathway utilised in the present invention from pathways described previously (discussed below).

By “altering the sequence of a nucleic acid molecule” is meant that the constituent nucleotide components of a nucleic acid molecule are changed in some way. Examples of alterations include the insertion, deletion or substitution of one or more constituent nucleotides in the target nucleic acid molecule, such as the introduction of a point mutation or creation of altered protein reading frames. Concerted combinations of insertions, deletions, and substitutions are also possible.

There is no restriction to the type of alteration event to which the present application is applied, although the most obvious applications include those which are extremely difficult or time consuming using approaches that are currently available. Examples include the precise modification of endogenous nucleic acid molecules in any species, such as yeast chromosomes, mouse embryonic stem cell chromosomes, C. elegans chromosomes, Arabidopsis and Drosophila chromosomes, human cell lines, viruses and parasites, or exogenous molecules such as plasmids, yeast artificial chromosomes (YACs) and human artificial chromosomes (HACs).

The first nucleic acid molecule, or replacement nucleic acid molecule, may be circular or linear, but is preferably a linear DNA or RNA molecule. Examples include single-stranded DNA or RNA, in either orientation, 5′ or 3′. Annealed oligonucleotides may also be used, either with blunt ends, or possessing 5′ or 3′ overhangs. Preferably, single-stranded oligonucleotides are used, most preferably, single-stranded deoxyribonucleotides. First nucleic acid molecules carrying a synthetic modification can also be used.

It should be noted that the replacement nucleic acid molecule is not necessarily a single species of nucleic acid molecule. For example, it is possible to use a heterogenous population of nucleic acid molecules, for example, to generate a DNA library, such as a genomic or cDNA library.

The second nucleic acid molecule is also referred to herein as the target nucleic acid molecule. A number of different types of nucleic acid molecule may be targeted using the method of the invention. Accordingly, intact circular double-stranded nucleic acid molecules (DNA and RNA), such as plasmids, and other extrachromosomal DNA molecules based on cosmid, P1, BAC or PAC vector technology may be used as the second nucleic acid molecule according to the invention described above. Examples of such vectors are described, for example, by Sambrook and Russell (Molecular Cloning, Third Edition (2000), Cold Spring Harbor Laboratory Press) and loannou et al. (Nature Genet. 6 (1994), 84-89) and the references cited therein.

The second nucleic acid molecule may also be a host cell chromosome, such as, for example, the E. coli chromosome. Alternatively, a eukaryotic host cell chromosome (for example, from yeast, C. elegans, Drosophila, mouse or human) or eukaroytic extrachromosomal DNA molecule such as a plasmid, YAC and HAC can be used. Alternatively, the target nucleic acid molecule need not be circular, but may be linear. Preferably, the second nucleic acid molecule is a double-stranded nucleic acid molecule, more preferably, a double-stranded DNA molecule.

It should be noted that either the first nucleic acid molecule or the second nucleic acid molecule should contain a selectable marker and an origin of replication. In this way, the selectable marker and/or origin of replication may be incorporated into the target nucleic acid molecule by repair recombination, in order that the nucleic acid molecule may be selected, and propagated in the host cell.

In the case that the first, but not the second nucleic acid molecule carries an origin plus selectable marker gene, the method of the invention may utilise the methods for nucleic acid subcloning as described by Zhang et al., Nature Biotech 18 (2000), 1314-1317; also see International patent application WO01/04288). An annealing protein, whether in the presence of RecE/Redα or not, may also effect such nucleic acid subcloning using either single-stranded first and/or second nucleic acid molecules, or double-stranded nucleic acid molecules, or any combination of a single-stranded and a double stranded riucleic acid molecule.

The first nucleic acid molecule should possess at least two regions of sequence homology with regions of sequence on the second nucleic acid molecule. By “homology” is meant that when the sequences of the first and second nucleic acid molecules are aligned, there are a number of nucleotide residues that are identical between the sequences at equivalent positions. Degrees of homology can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Such regions of homology are preferably at least 9 nucleotides each, more preferably at least 15 nucleotides each, more preferably at least 20 nucleotides each, even more preferably at least 30 nucleotides each. Particularly efficient recombination events may be effected using longer regions of homology, such as 50 nucleotides or more. Preferably, the degree of homology over these regions is at least 40%, 50%, 60%, 70%, 80%, 90%, 95%, 98% or 99% or more identity, as determined using BLAST version 2.1.3 using the default parameters specified by the NCBI (the National Center for Biotechnology Information; http:flwww.ncbi.nlm.nih.gov/) [Blosum 62 matrix; gap open penalty=11 and gap extension penalty=1].

The regions of sequence homology may be located on the first nucleic acid molecule so that one region of homology is at one end of the molecule and the other is at the other end. However, one or both of the regions of homology may also be located internally. The two sequence homology regions should thus be tailored to the requirements of each particular experiment. There are no particular limitations relating to the position for the two sequence homology regions located on the second DNA molecule, except that for circular double-stranded DNA molecules, the repair recombination event should not abolish the capacity to replicate. As the skilled reader will appreciate, the sequence homology regions can be interrupted by non-identical sequence regions, provided that sufficient sequence homology is retained to allow the repair recombination reaction to occur. By including in the first nucleic acid molecule, sequence homology arms that span regions of non-identical sequence compared to the second nucleic acid molecule, mutations such as substitutions, (for example, point mutations), insertions and/or deletions may be introduced into the second nucleic acid molecule.

Suitable phage annealing proteins for use in the invention (as known at the time of writing) include RecT (from the rac prophage), Redβ (from phage λ), and Erf (from phage P22). The identification of the recT gene was originally reported by Hall et al., (J.Bacteriol. 175 (1993), 277-287). The RecT protein is known to be similar to the λ bacteriophage β protein or Redβ (Hall et al. (1993), supra; Muniyappa and Radding, J.Biol.Chem. 261 (1986), 7472-7478; Kmiec and Holloman, J.Biol.Chem.256 (1981), 12636-12639). The Erf protein is described by Poteete and Fenton, (J Mol Biol 163 (1983), 257-275) and references therein. Erf is functionally similar to Redβ and RecT (Murphy et al., J Mol Biol 194 (1987), 105-117), and in some cases can substitute for the lambda phage recombination system (Poteete and Fenton, Genetics 134 (1993), 1013-1021). The Genbank ID for Erf is X05268 (VO1152). The sequences of RecT and Redβ are included herein as SEQ ID No. 1 (RecT) and SEQ ID No. 2 (Redβ).

The invention also includes the use of functional equivalents of the molecules that are explicitly identified above as RecT, Redβ and Erf, provided that the functional equivalents retain the ability to mediate recombination, as described herein. Such functional equivalents include homologues of elements of recombination systems that are present in bacteriophages, including but not limited to large DNA phages, T4 phage, T7 phage, small DNA phages, isometric phages, filamentous DNA phages, RNA phages, Mu phage, P1 phage, defective phages and phagelike objects, as well as the functional homologues of elements of recombination systems that are present in viruses, including but not limited to any virus which belongs to any of the following groups: plant viruses, insect viruses, yeast viruses, fungi viruses, parasitic micro-organism viruses, picornaviridae, enteroviruses, polioviruses, coxsackieviruses, echoviruses, rhinoviruses, all hepatitis viruses, caliciviridae, Norwalk group of viruses, astroviridae, astroviruses, togaviridae, alphaviruses, rubella virus, flaviviridae, flaviviruses, pestiviruses, coronaviridae, coronaviruses, lactate dehydrogenase-elevating virus and related viruses, rhabdoviridae, rhabdoviruses, filoviridae, Marburg viruses, ebola viruses, paramyxoviridae, parainfluenza viruses, mumps virus, measles virus, respiratory syncytial virus, orthomyxoviridae, orthomyxoviruses, bunyaviridae, arenaviridae, arenaviruses, reoviridae, reoviruses, rotaviruses, orbiviruses, coltiviruses, retroviridae, human T-cell leukemia virus, human immunodeficiency virus, lentiviruses, papoviridae, polyomavirinae, polyomaviruses, papillomavirinae, papillomaviruses, adenoviridae, adenoviruses, parvoviridae, parvoviruses, herpesviridae, herpes simplex viruses, Epstein-Barr virus, cytomegaloviruses, Varicella-Zoster virus, Human Herpesvirus, Cercopithecine Herpes Virus, B Virus, poxviridae, poxviruses, hepadnaviridae, and unclassified agents such as hepatitis Delta virus, and hepatitis E virus (Fields Virology, Third Edition, edited by B. N. Fields, D. M. Knipe, P. M. Howley, et al. Lippincott—Raven Publishers, Philadelphia Pa. USA (1996)).

Of course, as and when additional, functionally equivalent annealing proteins are discovered, for example, as a result of genome sequencing projects of other coliphages and lambdoid phages, it is envisaged that these annealing proteins will be equally suitable to those that are explicitly recited above.

Specific examples of functional equivalents of phage annealing proteins useful according to the invention include RAD52, forms of which are found in various organisms (see Passy et al., Proc Natl Acad Sci USA 96 (1999), 4279-4284) and Sepember 1 (Kolodner et al., Mol Microbiol 11 (1994), 23-30). Further examples of functional equivalent molecules include RecT, Redβ or Erf proteins that comprise amino acid substitutions, insertions and/or deletions from the wild type sequence, provided that these changes do not adversely affect the function of the annealing protein in mediating repair recombination as described herein. Such functional equivalents will preferably possess an amino acid sequence identity of at least 20%, preferably, of at least 40%, 50%, 60%, 70%, 80%, 90%, 95%, 98% or 99% or more with the wild type sequences that are depicted in the GenBank locations referenced above [as determined using BLAST version 2.1.3 using the default parameters specified by the NCBI].

Also included as functional equivalents are fragments of the RecT, Redβ and Erf proteins, such as truncated variants, and fusion proteins of which the sequence of a RecT, Redβ or Erf protein forms a part, that retain the ability to mediate homologous or repair recombination (for example, see Muyrers et al., Genes Dev 14 (2000), 1971-1982). It is considered that the identification of such functional equivalents is within the ability of the skilled addressee. For example, functional Erf truncation mutants have been identified (Poteete and Fenton, J Mol Biol 163 (1983), 257-275; Poteete et al., J Mol Biol 171 (1983), 401-418; Fenton and Poteete, Virology 134 (1984), 148-160).

Also included as functional variants are annealing protein variants that have been optimised and/or evolved, through, for example DNA shuffling (Stemmer, W. P. Nature 370, 389-91 (1994)), or Substrate-linked directed evolution (SliDE, see co-owned, co-pending United Kingdom patent application GB 0029375.3).

In order that recombination between the nucleic and molecules may be effected according to the invention, the first, replacement nucleic acid molecule must be brought into contact with the second, target nucleic acid molecule in the presence of a phage annealing protein, or a functional equivalent or fragment thereof.

The method of the invention may be effected, in whole or in part, in a host. Suitable hosts include cells of many species, including viruses and parasites, prokaryotes and eukaryotes, although bacteria, such as gram negative bacteria are a preferred host. More preferably, the host cell is an enterobacterial cell, such as a Salmonella, Klebsiella, Bacillus, Neisseria or Escherichia coli cell (the method of the invention works effectively in all strains of E. coli that have been tested). It should be noted, however, that the method of the present invention is also suitable for use in eukaryotic cells or organisms, such as fungi, plant or animal cells, as well as viral and parasitic cells and organisms. The system has been demonstrated to function well in mouse ES cells and there is no reason to suppose that it will not also be functional in other eukaryotic cells.

One aspect of the invention thus provides a method for altering the sequence of a nucleic acid molecule, comprising the steps of:

a) providing a host containing a phage annealing protein or a functional equivalent or fragment thereof;

b) contacting in said host, a first nucleic acid molecule, with a second nucleic acid molecule that comprises at least two regions of sequence homology with regions on the first nucleic acid molecule, under conditions suitable for repair recombination to occur between said first and second nucleic acid molecules; and

c) selecting a host in which repair recombination between said first and second nucleic acid molecules has occurred.

In prokaryotic hosts, the method may include the proviso that the RecE/Redα protein is not present during the course of the sequence alteration reaction. Preferably, the host cell used for repair recombination can be any cell in which a RecT, Redβ or Erf protein, or a functional equivalent or fragment thereof, is expressed. For example, the host cell may comprise the recT, redβ or erf gene located on the host cell chromosome or on a non-chromosomal nucleic acid molecule, such as a vector, optionally expressed from a promoter, such as the regulatable arabinose-inducible BAD or lac promoters or the strong constitutive promoter EM-7. Alternatively, RecT, Redβ or Erf may be expressed from a mRNA which is introduced with the first and, potentially, the second nucleic acid molecule. The repair recombination reaction faithfully integrates the replacement nucleic acid sequence. For example, in E. coli, all recombined molecules are proof-read by the endogenous replication and repair systems. As a result, the fidelity of sequence reproduction is extremely high.

In the system that is described here, the expression of the phage annealing protein or functional equivalent or fragment thereof may be controlled by a regulatable promoter. In this manner, the recombinogenic potential of the system is only elicited when required and, at other times, possible undesired recombination reactions are limited. Since many undesired recombination reactions occur through homologous recombination by double strand break repair (Muyrers et al., Genes Dev 14 (2000), 1971-1982; Zhang et al., Nature Biotech 18 (2000), 1314-1317), and therefore require the expression of both components of a phage protein pair (Muyrers et al., Genes Dev 14 (2000), 1971-1982), the risk of such unwanted recombination is greatly lowered in the presence of the annealing protein only. Moreover, given the independence of the system described here on the presence of RecA, this risk is further reduced by carrying out the method in a host cell in which no RecA is expressed.

As discussed above, the second nucleic acid molecule (the target nucleic acid molecule) may be a circularised or linear molecule, and may thus be expressed transiently or permanently in the host cell in this aspect of the invention, for example, from the chromosome or from an extrachromosomal element. The first nucleic acid molecule (the replacement nucleic acid molecule) may also be derived from any source, but, in this embodiment of the invention, will need to be introduced into the host cell in order for the recombination reaction to take place effectively. For example, the replacement nucleic acid molecule may be synthesized by a nucleic acid amplification reaction such as a PCR reaction, for example, in which both of the DNA oligonucleotides used to prime the amplification contain, in addition to sequences at the 3′-ends that serve as a primer for the amplification, one or the other of the two homology regions. Using oligonucleotides of this design, the nucleic acid product of the amplification can be any nucleic acid sequence suitable for amplification and will additionally have a sequence homology region at each end.

The method of the invention may comprise the contacting of the first and second nucleic acid molecules in vivo. In one embodiment, the first nucleic acid molecule may be transformed into a host cell that already harbours the second nucleic acid molecule. In a different embodiment, the first and second nucleic acid molecules may be mixed together in vitro before their co-transformation into the host cell. Of course, one or both of the species of nucleic acid molecule may be introduced into the host cell by any means, such as by transfection, transduction, transformation, electroporation and so on. For bacterial cells, the preferred method of transformation or cotransformation is electroporation.

The invention may be initiated entirely in vitro, without the participation of host cells or the cellular recombination machinery. Phage annealing proteins such as RecT are able to form complexes in vitro between the protein itself, an oligonucleotide molecule and a double-stranded nucleic acid molecule (Noirot and Kolodner, J Biol Chem 273 (1998), 12274-12280). One example of such a complex is that formed between RecT, a ssDNA oligonucleotide and an intact circular plasmid. Such complexes lead to the formation of complexes that are herein termed “joint molecules” (consisting, in this example, of the plasmid and the ssDNA oligonucleotide). Such joint molecules have been found to be stable after removal of the phage annealing protein. The formation of stable joint molecules has been found to be dependent on the existence of shared homology regions between the ssDNA oligonucleotide and the plasmid.

The potential of RecA to make joint molecules in vitro has already been exploited to allow the isolation of desired DNA strategies from a pool, for example in RecA-assisted-cloning (Ferrin and Camerini-Otero, Proc Natl Acad Sci USA 95 (1998), 2152-2157, for review see Ferrin, Methods Mol Biol 152 (2000), 135-147) and in RecA-mediated affinity capture (Zhumabayeva et al., Biotechniques 27 (1999), 834-840). This capacity of RecA has also been used for other tasks, such as RecA-assisted restriction endonuclease (RARE) cleavage (Ferrin and Camerini-Otero, Science 254 (1991), 1494-1497). However, no description or application exists to date in which recombination is initiated in vitro by a phage annealing protein and is completed in vivo, in the absence of any exogenously added protein, to result in a specifically modified nucleic acid molecule.

It is proposed herein that so-called “joint molecules” as described above may be used directly to mediate recombination in a host cell, where the host cell does not need to express any phage annealing protein whatsoever. This aspect of the invention provides a method for altering the sequence of a nucleic acid molecule, said method comprising the steps of:

a) exposing a first nucleic acid molecule to a phage annealing protein, or a functional equivalent or fragment thereof, in the presence of a second nucleic acid molecule, to generate a joint molecule, wherein said first and second nucleic acid molecule share at least two regions of sequence homology;

b) incubating said joint molecule under conditions suitable for repair recombination to occur between said first and second nucleic acid molecules; and

c) selecting a nucleic acid molecule whose sequence has been altered so as to include sequence from said second nucleic acid molecule.

The method may include the proviso that the RecE/Redα protein is not present during the course of a sequence alteration reaction that is carried out in vivo in a prokaryotic cell.

According to this aspect of the invention, joint molecules may be used to increase the efficiency of a recombination event.

It is also proposed herein that a nucleic acid molecule (for example a ssDNA oligonucleotide or a dsDNA molecule) that is coated by a phage annealing protein, or functional equivalent or fragment thereof, herein referred to as a “coated molecule”, is able to recombines with higher efficiency compared to a ‘naked’, uncoated nucleic acid molecule. This has important applications in techniques such as the engineering of exogenous and endogenous nucleic acid molecules in a number of species, including both prokaryotes and eukaryotes (including yeast, mouse, plants, Archae, Human cells, C. elegans, Drosophila, X. laevis and so on), as well as in viruses and parasites. Examples of exogenous nucleic acid molecules (first, replacement nucleic acid molecules as described herein) that may be recombined in this way include transposons, HACs, YACs, plasmids, whilst one preferred example of endogenous nucleic acid molecules (second, target nucleic acid molecules as described herein) is a chromosome.

This aspect of the invention therefore also provides the use of a phage annealing protein or a functional equivalent or fragment to increase the efficiency of a homologous or repair recombination event (for example, in DNA engineering or subcloning (see Zhang et al, Nature Biotech 18 (2000), 1314-1317). For example, prior to the introduction of a first, replacement nucleic acid molecule (which may be single-stranded or double-stranded) into a host cell, the replacement nucleic acid molecule may be incubated in the presence of the phage annealing protein, or functional equivalent or fragment, in vitro. The nucleic acid preparation may then be partially or totally purified from the annealing protein and transformed into a host cell where the recombination event may be effected. By using such coated molecules rather than naked molecules, the efficiency of any of the applications of the described activity can be enhanced significantly.

One embodiment of this aspect of the invention provides the use of an isolated complex of a phage annealing protein, or a functional equivalent or fragment thereof and a first nucleic acid molecule as a template for repair recombination processing, leading to the formation of recombinant molecules in a host cell that does not need to express any phage annealing protein whatsoever.

A further embodiment of this aspect of the invention provides the use of an isolated complex of a phage annealing protein, or a functional equivalent or fragment thereof, a first nucleic acid molecule, and a second, double-stranded DNA molecule (a joint molecule; Noirot and Koldner, J Biol Chem 273 (1998), 12274-12280), as a template for repair recombination processing, leading to the formation of recombinant molecules in a host cell that does not need to express any phage annealing protein whatsoever.

Delivery of coated or joint molecules to the host cell (which in many cases contains the target molecule) can be of several types: transformation, transfection, electroporation, etc (also eukaryotic delivery techniques), or by using a phage annealing protein that carries a tag which allows it to cross the cell wall, such as the TAT (Nagahara et al., Nature Med. 4 (1998), 1449-1452; Schwarze et al., Science 285 (1999), 1569-1572) or kFGF tag (Delli Bovi et al., Cell 50 (1987), 729-737; Yoshida et al., Proc. Natl. Acad. Sci. USA 84 (1987), 7305-7309; Peters et al., Proc. Natl. Acad. Sci. USA 86 (1989) 5678-5682).

In all of the aspects of the invention that are described above, for initiation of recombination in vitro, only a phage annealing protein and one or two nucleic acid moleculetypes are needed. For further steps, a host is presently needed in order to provide the proteins necessary for homologous or repair recombination reaction to proceed (this may no longer be the case when the mechanisms of homologous and/or repair recombination have been elucidated). In prokaryotes, the proteins necessary for homologous recombination to occur are likely to be similar to the proteins that are functional in the downstream processes of homologous recombination in the RecA pathway. Such proteins include, for example, proteins that can perform branch migration and resolution and DNA replication. Other likely proteins include those involved in DNA repair (see Trends in Bioch Sci 20 (1995). Several candidate proteins are already known (for review, see Kowalczykowski et al., Microbiol Rev 58 (1994),401-465; Trends in Bioch Sci 20 (1995)).

Particular examples of prokaryotic host cells in which the activity described herein has been demonstrated to occur include strains JC5519 (Willets and Clark, J Bacteriol 100 (1969), 231-239); JC8679 and JC9604 (Clark, Genetics 78 (1974), 259-271); DK1 (New England Biolabs); and DH10B (Gibco BRL).

In eukaryotes, functional homologues of prokaryotic proteins are known to exist, although this particular function has not been described before. Additional eukaryotic-specific factors may also be of importance. Suitable eukaryotic cells for the method of the invention include those in which DNA engineering by homologous recombination is known to be feasible, including, for example, most S. cerevisae strains, mouse ES cells (such as E14 and R1; see Joyner, Gene Targeting, a practical approach, (2000) second edition, Oxford University Press Inc. New York) and certain somatic cell lines such as BT-40. Moreover, any cells or species which contain functional pathways for DNA repair (which include most cells; for example, see Stucki et al., Prog Nucleic Acid Res Mol Biol 65 (2000), 261-298; Hansen and Kelley, J Pharmacol exp Ther 295 (2000), 1-9) are likely to be suitable.

It is a great strength of the method of the invention that no complex selection steps are necessary to select for recombined molecules.

The efficiency of the methods of recombination that are described herein is such that several selection methods become feasible for genetic engineering, and further allows the manipulation of a nucleic acid sequence in a single step.

However, after contacting the first and second nucleic acid molecules under conditions which favour repair recombination between the two molecular species, one or more nucleic acid molecules must be selected that represent species in which repair recombination between replacement and target nucleic acid molecules has occurred. This procedure can be carried out by several different methods, as will be clear to the skilled reader. Preferably, selection is using PCR, although hybridisation reactions, using techniques of blotting, or using assays, may also be used (see Sambrook and Russell; loc. sit.). Despite the high efficiency of the method of the invention, there may be occasions when selectable gene steps may be included in the methodology in order to enhance the efficiency of the method, including methods of antibiotic selection, and selection using site-specific recombinases. Examples of suitable selection methods are described, for example, in International patent application WO99/29837.

The discovery of the formation of isolated complexes (joint molecules and coated molecules) comprising a phage annealing protein, an oligonucleotide and, in the case of a joint molecule, a double-stranded nucleic acid molecule (Noirot and Kolodner, J Biol Chem 273 (1998), 12274-12280) has important ramifications for other technologies for which such properties are extremely useful. One example is discussed above, namely increasing the efficiency of repair and/or homologous recombination between nucleic acid molecules that have been contacted by a phage annealing protein.

A still further aspect of the invention provides the selection of a desired nucleic acid molecule from a mixture of nucleic acid molecules. By designing an oligonucleotide molecule that possesses a complementary sequence to the sequence of the nucleic acid molecule of interest, and incubating this oligonucleotide with a phage annealing protein, fragment or functional equivalent, under appropriate conditions for the formation of joint molecules as described above, the joint molecule complex that is formed may be used to separate the desired nucleic acid molecule from the mixture. The complex may be separated, for example, using affinity separation and selecting for the phage annealing protein or functional equivalent or fragment.

This aspect of the invention provides a method for the selection of a nucleic acid molecule of interest from a mixture of nucleic acid molecules, said method comprising the steps of:

a) exposing an oligonucleotide molecule that possesses a complementary sequence to the sequence of the nucleic acid molecule of interest with a phage annealing protein, or functional equivalent or fragment thereof, under conditions appropriate for the formation of a coated molecule or a joint molecule complex;

b) incubating the coated molecule or joint molecule complex with the mixture of nucleic acid molecules; and

c) selecting a nucleic acid molecule that is bound to a phage annealing protein or functional equivalent or fragment thereof.

One method for separation of joint or coated molecules may involve the use of an oligonucleotide that contains a synthetic tag. In order to generate recombinant molecules that contain the nucleic acid sequence of interest, the isolated joint or coated molecule may be introduced into a host cell. Due to the properties of joint and coated molecules generated using a phage annealing protein or functional equivalent or fragment thereof, the host cell does not need to express phage annealing protein for repair recombination to occur.

Besides the above-described applications in the engineering and subcdoning of nucleic acid molecules, nucleic acid molecules coated with phage annealing protein, or functional equivalents or fragments thereof, may be used in anti-sense strategies, for example, based on RNA binding and inhibition, blocking of mRNA, inhibition of translation by blocking rRNA, and blocking of RNA transport. Libraries of first (replacement) nucleic acid molecules may also be utilised for random or targeted anti-sense. Such a method is feasible in potentially any organism.

According to a still further aspect of the invention, there is provided a method for cloning a nucleic acid, utilising a method of altering the sequence of a nucleic acid molecule as described in any one of the aspects of the invention described above.

According to a still further aspect of the invention, there is provided a method for engineering the sequence of a nucleic acid molecule, comprising a method of altering the sequence of a nucleic acid molecule as described in any one of the aspects of the invention described above.

The invention will now be described in detail, with particular emphasis on repair recombination mediated by RecT and Redβ. It will be appreciated that modification of detail may be made without departing from the scope of the invention.

### BRIEF DESCRIPTION OF THE FIGURES

FIG. 1. Phage annealing proteins mediate recombination between an intact circular plasmid and a single stranded DNA oligonucleotide. (A) Repair recombination between an oligonucleotide and plasmid pGKneo* results in the restoration of the functional neo gene to create pGKneo, which can be selected for by growth on LB plates containing kanamycin. bla indicates the ampicillin resistance gene. (B) Repair recombination results in the addition of sequence to the intact circular plasmid. As in (A), recombination between pGKneoΔ, which was generated from pGKneo to contain a defective neo gene, and the oligonucleotide results in the restoration of the functional neo gene to create pGKneo. Again, this phage annealing promoted recombination event can be selected for by growth on LB plates containing kanamycin.

FIG. 2. A) Diagram of the recombination process. ssDNA oligonucleotides with different length of homology regions were co-electroporated with pGkneo* into a host strain expressing phage annealing proteins. (B) Recombination tested in JC5519, mediated by RecT (T), Redβ (β) or no exogenous protein (C). On the X-axis, the nt length of the homology regions present on the ssDNA oligonucleotides is given (the right and left homology regions are of the same length). The Y-axis states the normalised recombination efficiency.

FIG. 3. Increased amount of nucleotides that need to recombine from the oligonucleotide into the circular plasmid correlates with a decrease in recombination efficiency. (A) Diagram of the oligonucleotides and plasmids used in the recombination assay. (B) Normalised recombination efficiency achieved using the oligonucleotides and plasmids described in (A) and either RecT or Redβ to mediate the recombination reaction.

FIG. 4. Point mutations present in either homology region on the ssDNA oligonucleotide are not recombined into the circular plasmid and do not block recombination efficiency. (A) Diagram of the oligonucleotides and plasmid used in the recombination assay. (B) Normalised recombination efficiency achieved using the oligonucleotides described in (A), pGKneo* and either RecT or Redβ to mediate the recombination reaction, as indicated.

FIG. 5. ssDNA oligonucleotides containing terminal dideoxy residues are recombination proficient. (A) Diagram of the oligonucleotides and plasmid used in the recombination assay. (B) Normalised recombination efficiency achieved using the oligonucleotides described in (A), pGKneo* and either RecT or Redβ to mediate the recombination reaction, as indicated.

FIG. 6. Phage annealing protein mediated recombination can be used for chromosomal engineering. (A) Outline of the oligonucleotides and bacterial strain used. Both orientations of ssDNA oligonucleotides were used to repair the deficient neo gene present on the chromosome of JC5519neo* (JC5519 carrying the defective neo* gene on its chromosome) or JC5519neoΔ (JC5519 carrying the defective neoΔ gene on its chromosome). The shown oligonucleotides were electroporated into either JC5519neo* or JC5519neoΔ. These strains also expressed a phage annealing protein. (B) Normalised recombination efficiency achieved using the oligonucleotides shown in (A), and JC5519neo* or JC5519neoΔ expressing either RecT or Redβ to mediate the recombination reaction, as indicated. cmr indicates the chloramphenicol resistance gene.

FIG. 7. Phage proteins mediate recombination between a linearised dsDNA plasmid and a ssDNA oligonucleotide. (A) Diagram of the oligonucleotides and plasmid used in the recombination assay. The shown oligonucleotide was co-electroporated with the NcoI-linearised, mung bean nuclease treated pGKneo plasmid (to remove four nucleotides from the neo gene, see experimental protocol) into the JC5519 gene expressing a phage annealing protein. Selection pressure was exerted only for expression of the bla gene present on the linearised plasmid. After recombination, intact circular plasmids were obtained which contained the sequence originally present between the homology regions of the ssDNA oligonucleotide. These recombined plasmids thus contained a functional neo gene, (cells harbouring this plasmid were capable of growing on LB-plates containing kanamycin; data not shown). (B) Normalised recombination efficiency achieved using only the NcoI-linearised, mung bean nuclease treated pGKneo (a control for recircularisation without recombination), or NcoI linearised, mung; bean nuclease treated pGKneo plus the oligonucleotide shown in (A), in JC5519 expressing either RecT or Redβ to mediate the recombination reaction, as indicated.

FIG. 8. RecT can form a stable, homology-region dependent joint molecule between a ssDNA oligonucleotide and a plasmid which share sequence homology. Purified RecT was first incubated in vitro with the indicated ssDNA oligonucleotide (which, for detection purposes was ³²P end-labeled). In this step, the ssDNA oligonucleotide is coated by RecT. Then, either pGKneo* (which shares two homology regions with the oligonucleotide) or pBluescript (which shares no homology regions with the oligonucleotide) was added, followed by additional incubation. The reaction mixture was subsequently deproteinised, followed by agarose gel electrophoresis and detection of the readioactive signal. Only if the two DNA molecules share homology regions (pGKneo* and its partner ssDNA oligonucleotide), stable joint DNA molecules (consisting of pGKneo* and its partner ssDNA oligonucleotide) were formed (indicated by the arrow). Joint molecules were optimally formed at a RecT concentration of approximately 0.2 μg/μl. If no deproteinisation was carried out, RecT and the two involved molecules were found to be together in a high-molecular weight complex (data not shown).

FIG. 9. Schematic representation of pcDNA/PGK-neo*, pcDNA-redβ/PGK-neo* and pcDNA-recET/PGK-neo*.

FIG. 10: Schematic representation of the experiment performed in mouse ES cells (Example 9).

FIG. 11: Results of experiments detailed in Example 9.

FIG. 12: Schematic representation and results of the experiment detailed in Example 10.

FIG. 13: Schematic representation and results of the experiment detailed in Example 11.

### EXAMPLES

Note: All oligonucleotides were obtained from the EMBL oligonucleotide service.

### Example 1

Repair Recombination Using a Phage Annealing Protein

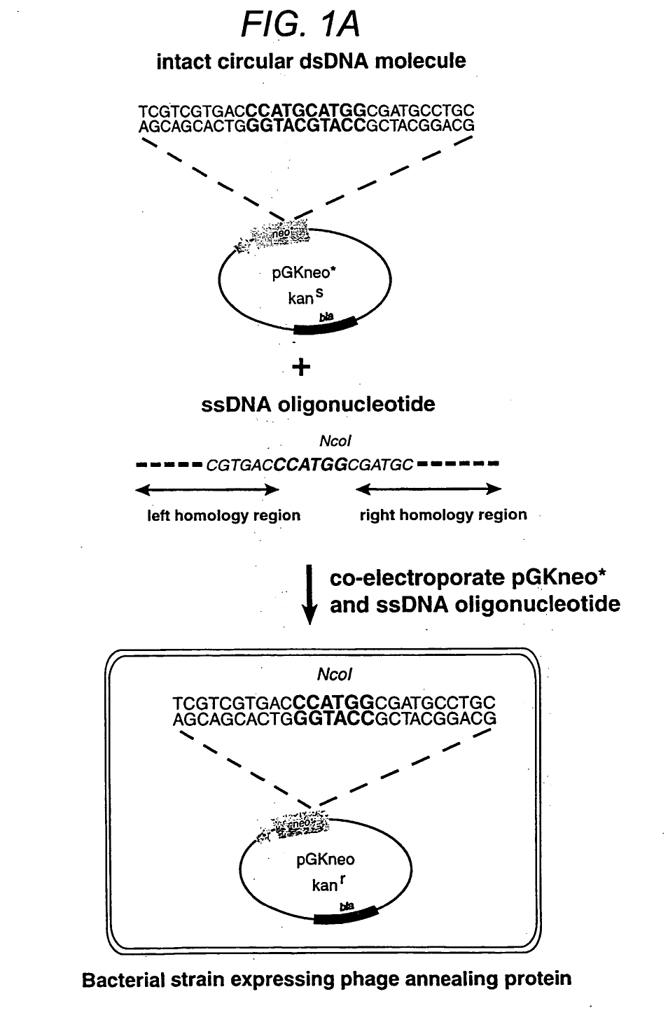

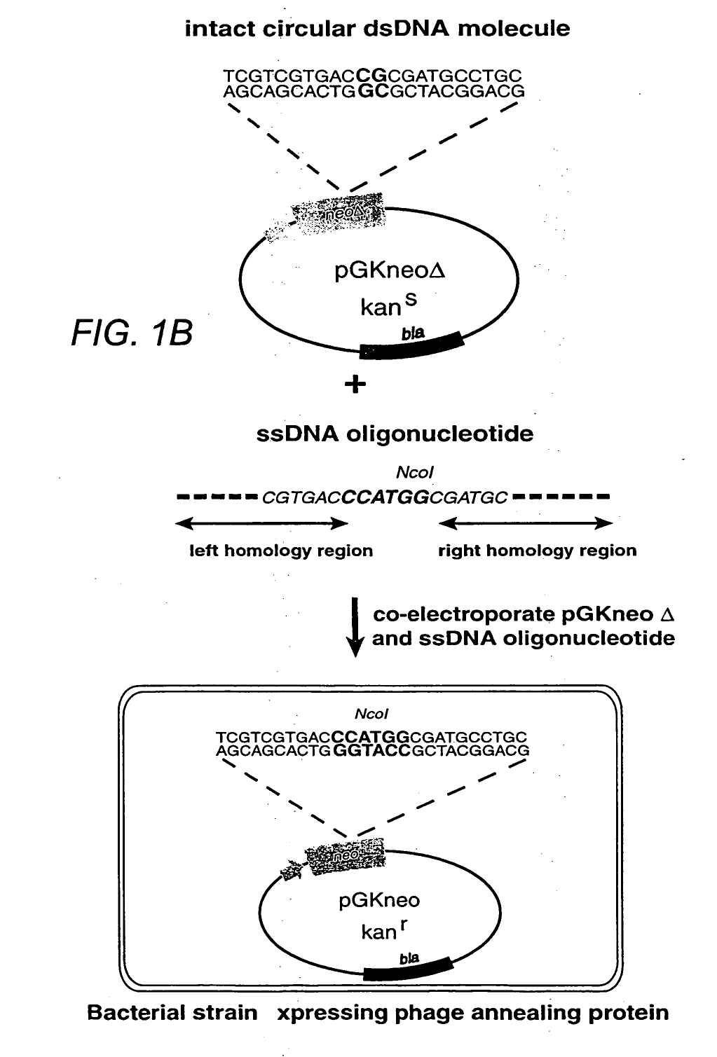

The recombination activity of phage annealing proteins to mediate repair recombination between two DNA molecules was initially found using the experiment of FIG. 1.

In the example shown in FIG. 1, regions of homology in a replacement oligonucleotide were chosen to flank a defective region in the neo gene present on an intact circular plasmid, pGKneo* or pGKneoΔ. These homology regions were also included in the oligonucleotide to flank the sequence that was originally present in the neo gene. In this example, pGKneo* was used, generated from pGKneo (Zhang Y., Muyrers J. P. P., Stewart A. F., unpublished data; sequence of pGKneo is given in SEQ ID No:3) to contain a defective neo gene. Host cells containing pGKneo* are therefore unable to grow in the presence of kanamycin selection. PGKneo* also carries the bla gene for selection by ampicillin. Through repair recombination between the intact circular plasmid and the oligonucleotide, mediated by a phage annealing protein, the defective neo gene may be repaired to generate a functional neo gene on pGKneo. This recombination event can be selected for by growth on LB-plates containing kanamycin.

The oligonucleotide (replacement nucleic acid molecule) used consists of a left homology region, the nucleotides that were originally present in the neo gene of pGKneo and a right homology region. pGKneo* and the oligonucleotide were co-electroporated into a host strain, usually JC5519 (Willetts and Clark, J Bacteriol 100 (1969), 231-239) that expresses a phage annealing protein. The sequences of oligonucleotides used herein are given in SEQ ID Nos: 4 and 5.

FIG. 1B shows repair recombination, resulting in the addition of sequence to the intact circular plasmid. As in FIG. 1A), recombination between pGKneoΔ, which was generated from pGKneo to contain a defective neo gene, and the oligonucleotide results in the restoration of the functional neo gene to create pGKneo. Again, this phage annealing-promoted recombination event can be selected for by growth on LB plates containing kanamycin.

To allow repair recombination, the two DNA molecules need to share two homology regions, stretches of shared DNA sequence that guide the recombination process to the correct region and through which repair recombination occurs. The sequence of these homology regions can be chosen freely, allowing DNA engineering at any position.

Experimental Protocol:

pGKneo* and pGKneoΔwere made from pGKneo by the following procedure: pGKneo was linearised with the NcoI restriction enzyme, which has a unique recognition site in the neo gene. To generate pGkneo*, the 5′overhangs of the NcoI digested pGkneo were filled in using Klenow and nucleotides according to the manufacturer's instructions (New England Biolabs), followed by ligation to generate an intact circular plasmid. To generate pGKneoΔ, the 5′overhangs of the NcoI digested pGKneo were removed using Mung Bean nuclease according to manufacturer's instructions (New England Biolabs), followed by ligation to generate an intact circular plasmid.

The intact circular plasmid and the oligonucleotide were co-electroporated into electrocompetent host cells. Only those electrocompetent host cells in which a phage annealing protein was expressed allowed repair recombination to generate a functional neo gene. Electrocompetent E. coli cells were prepared as described previously (Zhang et al., Nature Genet 20 (1998), 123-128; Muyrers et al., Nucd Acids Res 27 (1999), 1555-1557; Muyrers et al., Genes Dev 14 (2000), 1971-1982; Muyrers et al., EMBO R 1 (2000), 239-243; Muyrers et al., Genetic Engineering, Principles and Methods, J. K. Setlow Ed. 22 (2000), 77-98, Kluwer Academic/Plenum Publishers, NY; Zhang et al., Nature Biotech 18 (2000), 1314-1317; further information is available from http:H/www.embl-heidelberg.de/ExternalInfo/stewart/index.html). Briefly, 250 ml cultures of cells capable of expressing a phage annealing protein were started by 100-fold dilution of a saturated overnight culture into fresh LB medium. These cells were grown at 37 C to an OD₆₀₀ of 0.4 at which they were harvested. If necessary for expression of the phage annealing protein (i.e. in case the phage annealing protein gene was present on a pBAD24 based plasmid within the host cell) the cells were induced 1 hour prior to harvesting by adding L-arabinose (Sigma) to a final concentration of 0.1 %. The cells were harvested by centrifugation at 7000 rpm in a Sorvall SLA1500 rotor for 8 minutes at −3 C. The pellet was resuspended in 250 ml ice-cold 10% glycerol and centrifuged again (7000 rpm, 8 minutes, −3 C). This was repeated twice more, after which the cell pellet was suspended in an equal volume of ice-cold 10% glycerol. Aliquots (50 μl) were co-electroporated with the two DNA molecules (in the case of FIG. 1, an intact plasmid and an oligonucleotide). After electroporation on a BioRad Gene Pulser (2 mm cuvettes, 2.5 kV, 25 μF, 200 Ohm), the cells were incubated at 37 C for 1.5 hour with shaking and spread on antibiotic plates. The used concentration for kanamycin selection was 50 μg/ml; for ampicillin selection 50 μg/ml.

Host cells which supported the repair recombination reaction either expressed a phage annealing protein from the endogenous chromosome or from a plasmid which allows the constitutive or inducible expression of (at least) a phage annealing protein. Phage annealing protein genes were expressed inducibly from the promoter present on pBAD24, which allows inducible expression by addition of L-arabinose (Guzman et al.,J Bacteriol 177 (1995), 4121-4130).

Using the experiment described in FIG. 1A, several recombination pathways and proteins were tested for their ability to support this type of repair recombination. The results of these experiments are summarised in Table 1, which shows the results of an assessment of several recombination pathways and proteins for their ability to mediate repair recombination between a single stranded oligonucleotide and an intact circular plasmid, as described in FIG. 1. Indicated are the name of the tested strain, the genotype of this strain, the recombination mediating pathways or proteins present in the strain and the normalised amounts of kanamycin resistant, pGKneo containing recombinants. In all the experiments done to generate the data of this table, ssDNA oligonucleotides containing left and right homology regions of 22 nts were used. In all experiments of this table, data represent an average of at least 2 independent experiments.

Details of the experiment are as described in Table 1. Strains were co-electroporated with 0.1 μg of pGKneo* plus 2.2 μg of an oligonucleotide consisting of a 22 nt left homology region, the nucleotides that were originally present in the neo gene of pGKneo and a right homology region of 22 nt. All pBAD-based constructs allow inducible expression of the indicated genes from the L-arabinose inducible promoter. Host cells containing such a construct were L-ara induced prior to harvesting, as described above. To be able to normalise the amount of recombinants obtained on LB plates containing kanamycin, the amount of colonies obtained by transforming a standard amount (0.5 ng) of pBR322 plasmid was determined for every competent cell preparation of every tested strain. The strain to strain variation of the amount of colonies thus obtained was used as a normalization factor. For LB-kanamycin selection, a concentration of 50 μg/ml was used.

It is apparent that only host strains that express a phage annealing protein (RecT, Redβ and Erf) were found to mediate the required recombination reaction. Furthermore, a phage annealing protein is required and sufficient to mediate the described activity. In contrast to ET recombination (see International patent application WO99/29837), the expression of the annealing protein alone suffices to exhibit the described activity. No other recombination pathway (RecA, RecA/RecBCD, RecF) was found to be capable of mediating the required recombination reaction. Also, in contrast to ET recombination, the expression of RecBCD did not impede the described activity.

### Example 2

Length of Homology Regions Necessary

To investigate the activity of the phage annealing proteins further, the relationship between the length of the homology regions and the efficiency of recombination was tested.

Following the experimental setup of FIG. 1, a set of oligonucleotides in which the homology regions were chosen to vary from 5 up to 50 nucleotides were tested for their ability to repair the defective neo gene of pGKneo*. Phage proteins were inducibly expressed from pBAD24-based plasmids containing the corresponding genes. Electrocompetent cells which inducibly express the indicated proteins were prepared as described above. Electroporation, selection and normalisation of the obtained recombination efficiencies were done using the protocol and conditions of FIG. 1 and Table 1.

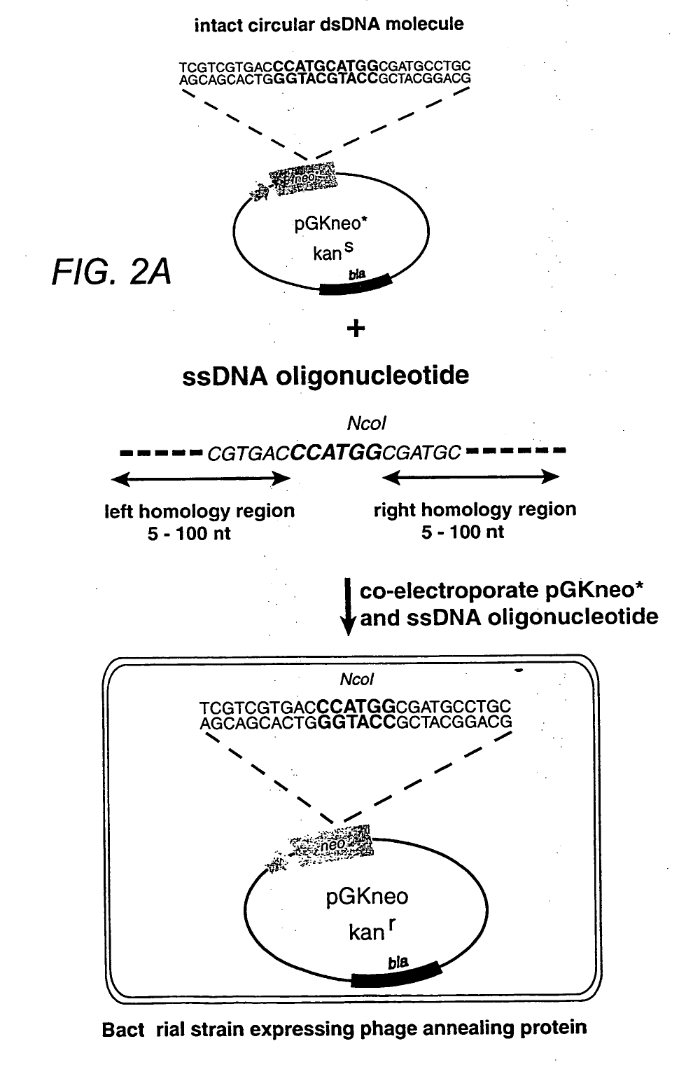

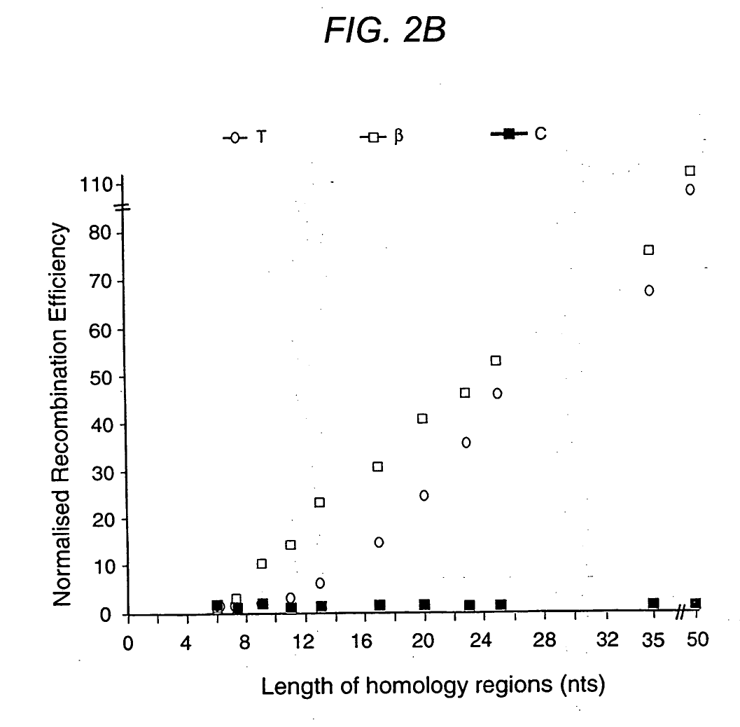

The results of this experiment are summarised in FIG. 2, which shows results relating to the relationship between homology region length and recombination efficiency.

Recombination was found to be detectable at very short homology regions of approximately 9 nucleotides. However, for both RecT and Redβ, increased length of homology region correlated with an increased recombination potential.

To determine whether two homology regions are strictly required for recombination to occur, oligonucleotides were offered for recombination which consisted of the nucleotides that can repair the defective neo gene of pGkneo*, and only one homology region (either left or right). Such oligonucleotides, containing only one homology region of variable length, were tested using the assay described in FIG. 1. The results are summarised in Table 2.

These results show that both for RecT and Redβ, recombination could only be detected when two homology regions were present on the single stranded oligonucleotide. Thus, two homology regions are strictly required for repair recombination mediated by a phage annealing protein.

Several aspects of the described recombination mechanism were studied in more detail, using the recombination assay described in FIG. 1A.

Table 3 indicates the lengths of the homology regions present on the ssDNA oligonucleotide, the orientation of the oligonucleotide (complementary to either the bottom or top strand of the defective neo gene of pGKneo*) and the normalised recombination efficiency achieved using either RecT or Redβ to mediate the recombination reaction, using the assay of FIG. 1. For this experiment, the indicated phage proteins were inducibly expressed in JC5519 from pBAD24-based plasmids containing the corresponding genes. Electrocompetent cells which inducibly express the indicated proteins were prepared as described above, as was electroporation, selection and normalisation of the obtained recombination efficiencies.

From these experiments, it was determined that the oligonucleotides used for recombination can be designed to be complementary to either strand of the defective neo gene of pGKneo* (see Table 3). However, a consistent difference was found in recombination efficiency, which was higher for oligonucleotides that are complementary to the bottom strand compared to oligonucleotides that were complementary to the top strand.

### Example 3

Recombination Efficiency in Relation to the Number of Nucleotides that Need to Recombine

The experimental details of this experiment were as follows. Four types of pGKneo-derived plasmids were constructed to each contain a sequence deletion in the neo gene of varying length, rendering the neo gene defective in each of these plasmids. In pGKneoΔ, 4 nucleotides are deleted from within the neo gene (see FIG. 1B), in pGKneoΔ15, 15 nucleotides were deleted, in pGKneoΔ33, 33 nucleotides were deleted; and in pGKneoΔ60, 60 nucleotides were deleted. Each of these plasmids was recombined with an oligonucleotide that contains the missing sequence flanked by homology regions of 25 nucleotides. Thus, every pGKneoΔ plasmid variant was co-electroporated with its own specific oligonticleotide which contained the missing sequence of that plasmid type (4, 15, 33 or 60 nucleotides, depending on which plasmid was used), flanked by homology regions of 25 nts (the same for all plasmids used). For every recombination reaction, the length of the homology region was the same, namely 25 nts. To generate pGKneoΔ15, pGKneoΔ33 and pGKneoΔ60, pGK-neo was digested with NcoI which cuts uniquely in the neo gene. Sequence deletions were subsequently generated by incubation with Bal31 according to manufacturer's instructions (New England Biolabs). After Bal31 digestion, the obtained molecules were ligated to generate intact circular plasmids. The length of the generated sequence was determined by DNA sequencing. The indicated phage proteins were inducibly expressed in JC5519 cells from pBAD24-based plasmids containing the respective genes. Electrocompetent cells which inducibly expressed the indicated proteins were prepared as described above, as was electroporation, selection and normalisation of the obtained recombination efficiencies.

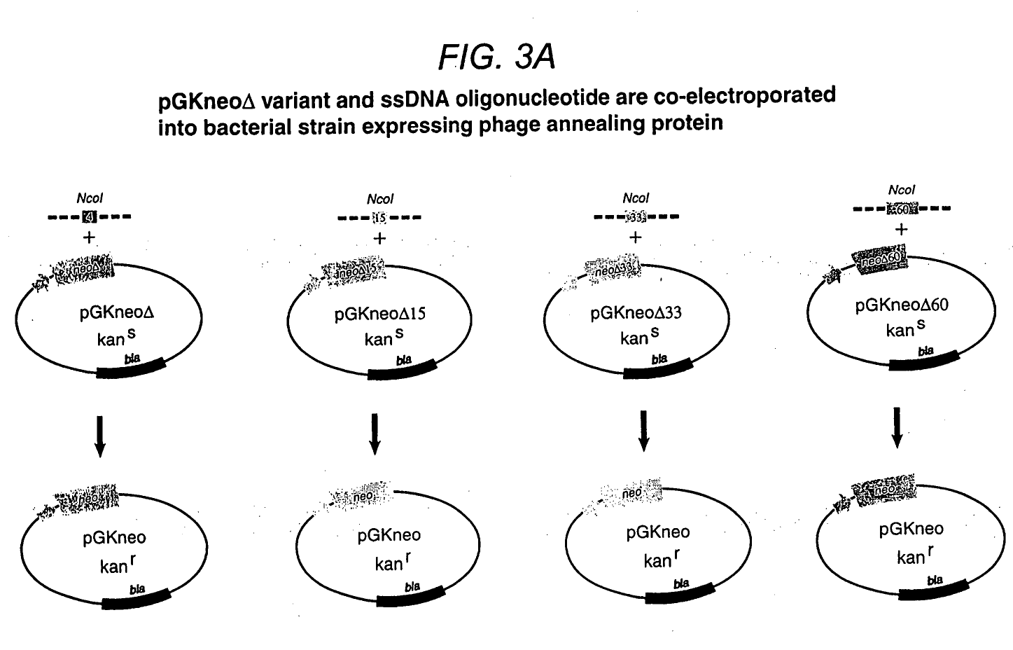

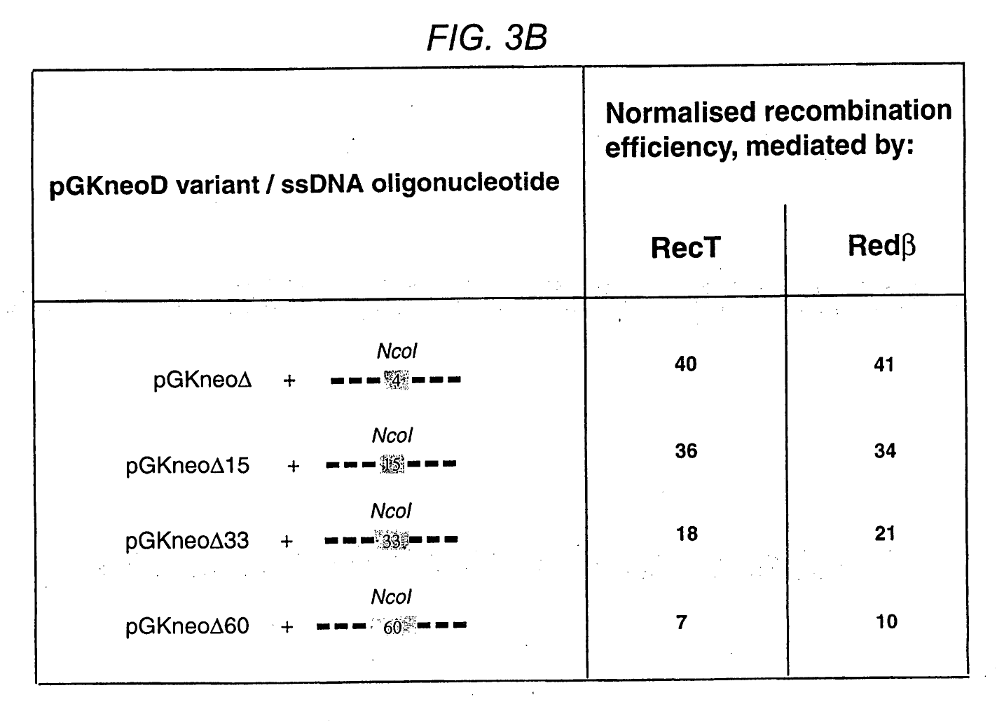

It was found that there is a negative correlation between the recombination efficiency and the amount of nucleotides which need to be recombined into a circular plasmid and which are present between the homology regions on the single stranded oligonucleotide (see FIG. 3, and legend).

### Example 4

Point Mutations Present in Homology Regions

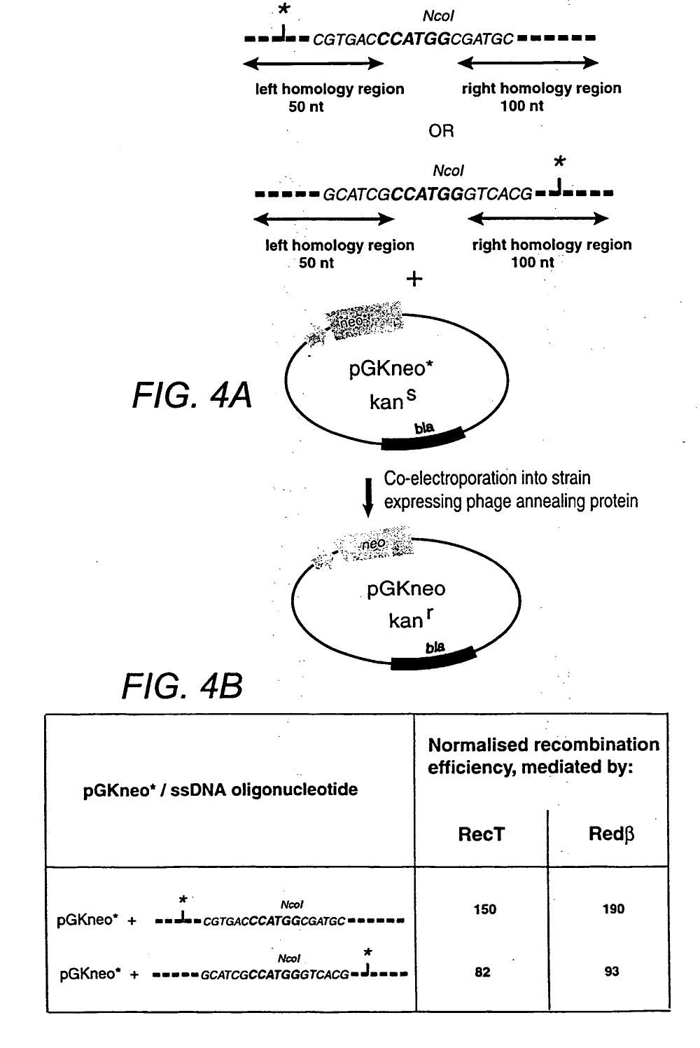

FIG. 4A shows a diagram of the oligonucleotides and plasmid used in the recombination assay. Both orientations of oligonucleotides were tested (complementary to the top strand, or to the bottom strand, see Table 3). Depending on which oligonucleotide was used, the point mutation is 5′ relative to the sequence that can repair the defective neo gene (as is the case in the oligonucleotide that is complementary to the bottom strand), or 3′ relative to the sequence that can repair the defective neo gene (as is the case in the oligonucleotide that is complementary to the top strand). The introduced point mutation, if recombined into the neo gene, introduces a silent mutation in the neo gene, thereby leaving the protein sequence and the function of the gene unaltered. These oligonucleotides were co-electroporated with the pGKneo* plasmid into the JC5519 gene expressing a phage annealing protein.

FIG. 4B presents the recombination efficiencies achieved. Of approximately 50 recombinant clones examined after recombination with a ssDNA oligonucleotide for each orientation, none had incorporated the point mutation present in the homology region into the recombinant product (data not shown).

This experiment demonstrated that a single point mutation present in either homology region is not recombined into the circular plasmid, and does not abolish the recombination reaction. The experimental procedures for this experiment were as follows. The indicated phage proteins were inducibly expressed in JC5519 from pBAD24-based plasmids containing the corresponding genes. Electrocompetent cells which inducibly express the indicated proteins were prepared as described above, as was electroporation, selection and normalisation of the obtained recombination efficiencies.

### Example 5

Single-Stranded DNA Oligonucleotides Containing Terminal Dideoxy Residues are Recombination Proficient

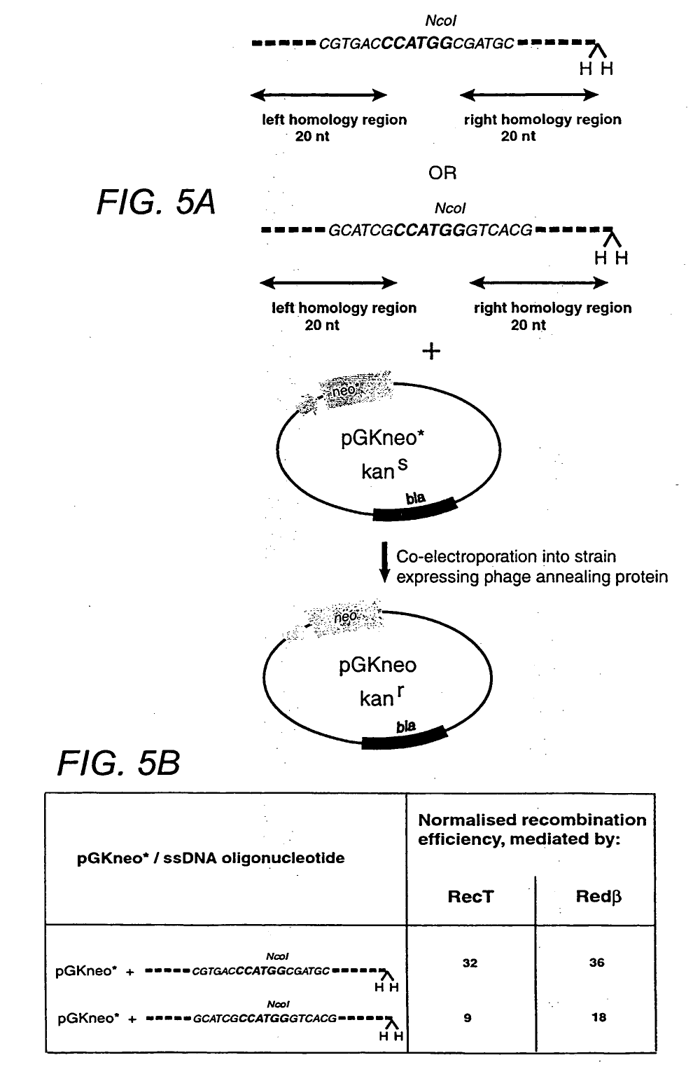

FIG. 5A shows a diagram of the oligonucleotides and plasmid used in the recombination assay. Both orientations of oligonucleotides were tested (complementary to the top strand, or to the bottom strand, see Table 3). In both orientations, the 3′terminus of the ssDNA oligonucleotide contains a dideoxy residue. These oligonucleotides were co-electroporated with the pGKneo* plasmid into the JC5519 gene expressing a phage annealing protein.

These experiments showed that the presence of a dideoxy residue at the very 3′ terminus of a single stranded oligonucleotide does not block recombination, regardless of the orientation of the single stranded DNA oligonucleotide (see FIG. 5). The experiment procedures for this experiment were as described above. The indicated phage proteins were inducibly expressed in JC5519 from pBAD24-based plasmids containing the corresponding genes.

### Example 6

Targeting of E. coli Chromosome

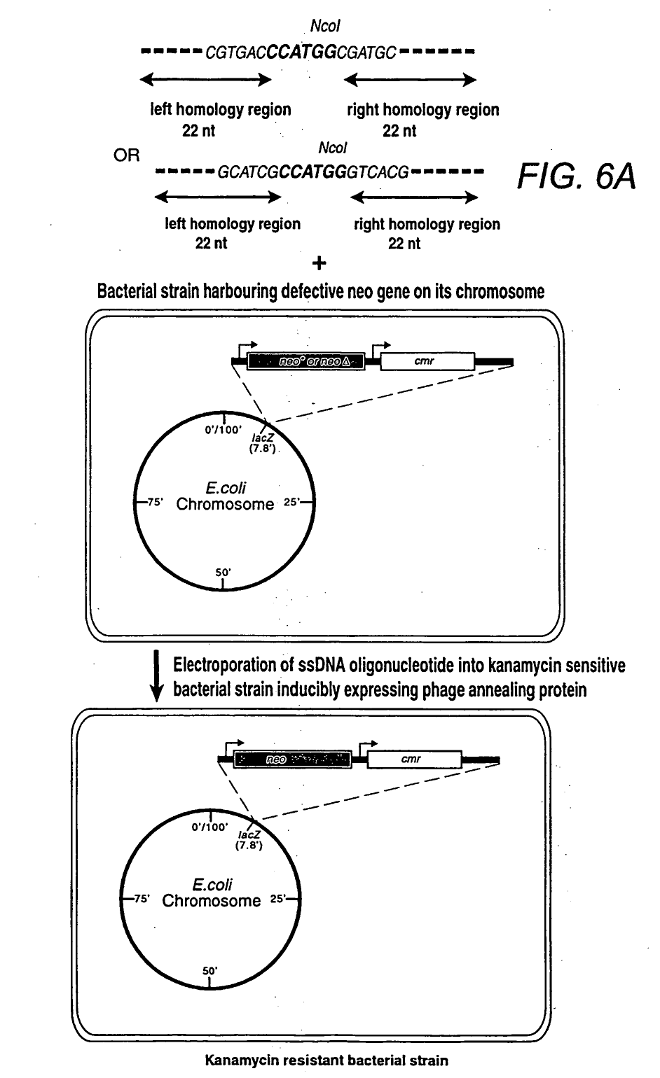

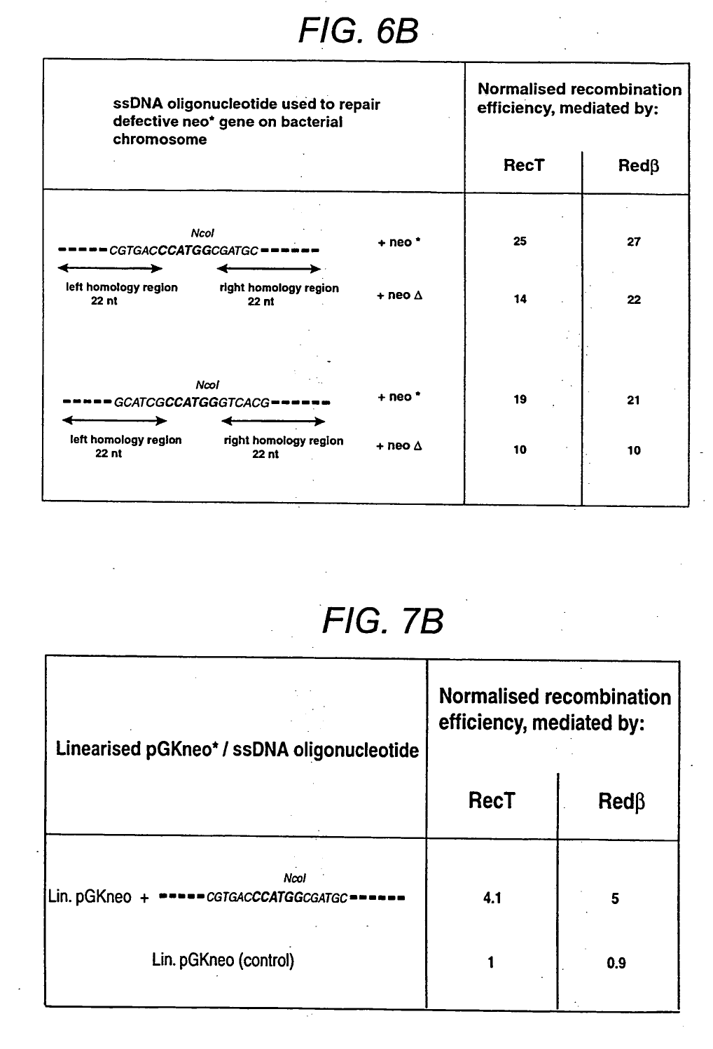

To determine whether the E. coli chromosome could be targeted through phage annealing protein mediated repair recombination with single stranded oligonucleotides, the assay described in FIG. 6 was performed.

The assay is in principle similar to the assay of FIG. 1. The phage proteins were inducibly expressed in JC5519 from pBAD24-based plasmids containing the corresponding genes (see FIG. 6). JC5519neo* and JC5519neoΔ were generated by ET recombination using the following procedure. First, a chloramphenicol resistance gene (cmr) and its promoter were cloned by ET recombination, 3′ of the defective neo gene of pGKneo* and pGKneoΔ. Then, a PCR fragment was generated to contain the neo* and cmr genes and their promoters (amplified from pGKneo* containing cmr), or to contain the neoΔ and cmr genes and their promoters (amplified from pGKneoΔ containing cmr). These PCR fragments also contained homology regions that allowed targeting of the fragment to the lacZ locus of JC5519. After targeting of JC5519 with the neo*-cmr, or the neoΔ-cmr cassette and selection on LB-plates containing chloramphenicol, JC5519neo*, respectively JC5519neoΔ, were obtained. The correct integration was confirmed by Southern analysis.

Electrocompetent cells which inducibly express the indicated proteins were prepared exactly as described in the legend to FIG. 1. Electroporation, selection and normalisation of the obtained recombination efficiencies were done using the protocol and conditions of FIG. 1 and Table 1. For chloramphenicol selection during the ET cloning of the defective neo gene onto the chromosome of JC5519, a concentration of 20 μg/ml was used. To select for correct recombinants with a functional neo gene, a concentration of 20 μg/ml was used.

In FIG. 6, the targeted sequence is thus the defective neo gene (taken from pGKneo* and from pGKneoΔ) which was placed on the chromosome of the JC5519 E. coli host strain by ET recombination. Competent cells expressing a phage annealing protein and containing the defective neo gene were prepared and electroporated with a ssDNA oligonucleotide which, by repair recombination, repaired the neo gene. Both orientations of a single stranded oligonucleotide (complementary to the top or bottom strand of the neo gene) were found to be functional in this assay, which argues against a recombination theory in which the single strand oligonucleotide functions directly as an Okazaki fragment to initiate replication.

### Example 7

Absolute Engineering Efficiency

To obtain the data shown above, the repair of the neo gene was utilised as a convenient system to score for recombination events. However, in most practical applications the sequence that is modified by repair recombination cannot be selected for using antibiotics. Therefore, it was desirable to determine the absolute efficiency of the described activity.

In order to do this, the same model system was used as is shown in FIG. 1A, and the number of colonies obtained was compared on two different selection plates. After co-electroporation of pGKneo* and the oligonucleotide (as shown in FIG. 1A) into a phage annealing protein expressing host cell, an equal amount of cells (in serial dilutions) were plated onto LB-plates containing only ampicillin, and in parallel onto LB-plates containing ampicillin plus kanamycin. On LB-plates containing ampicillin only, any cell that has been electroporated with the pGKneo* plasmid can form a colony. However, on LB-plates containing kanamycin plus ampicillin, only those cells in which recombination has taken place to restore a functional neo gene can survive.

The results of this experiment are summarised in Table 4, which shows that recombination between a single stranded oligonucleotide and an intact circular plasmid is highly efficient. A ssDNA molecule containing two homology regions of 50 nt each was co-lectroporated with pGKneo* in JC5519 expressing a phage annealing protein, as described in the assay of

FIG. 1. After electroporation and incubation at 37° C., an equal amount of cells were plated on LB-plates containing ampicillin and on LB-plates containing ampicillin plus kanamycin. The colony number obtained on the indicated LB-plate, using the indicated phage annealing protein, is shown. In all cases, the indicated phage proteins were inducibly expressed in JC5519 from pBAD24-based plasmids containing the corresponding genes. Electrocompetent cells which inducibly express the indicated proteins were prepared exactly as described above, as were electroporation, selection and normalisation of the obtained recombination efficiencies. Both for kanamycin and for ampicillin selection, a concentration of 50 μg/ml was used. Data shown presents the average value of 3 independent experiments.

From the ratio between the amount of colonies obtained on plates containing only ampicillin and the amount of colonies obtained on plates containing ampicillin plus kanamycin, it can be concluded that, depending on which phage annealing protein is used, approximately 1 in every 200 electroporated cells underwent repair recombination. This is a very workable number to allow selection methods that are not based on antibiotic selection to identify the desired recombinants from the total pool of electroporated cells. Such alternative selection methods include selective PCR based strategies, restriction enzyme analysis and colony hybridisation.

### Example 8

Recircularisation of Linearised Plasmids

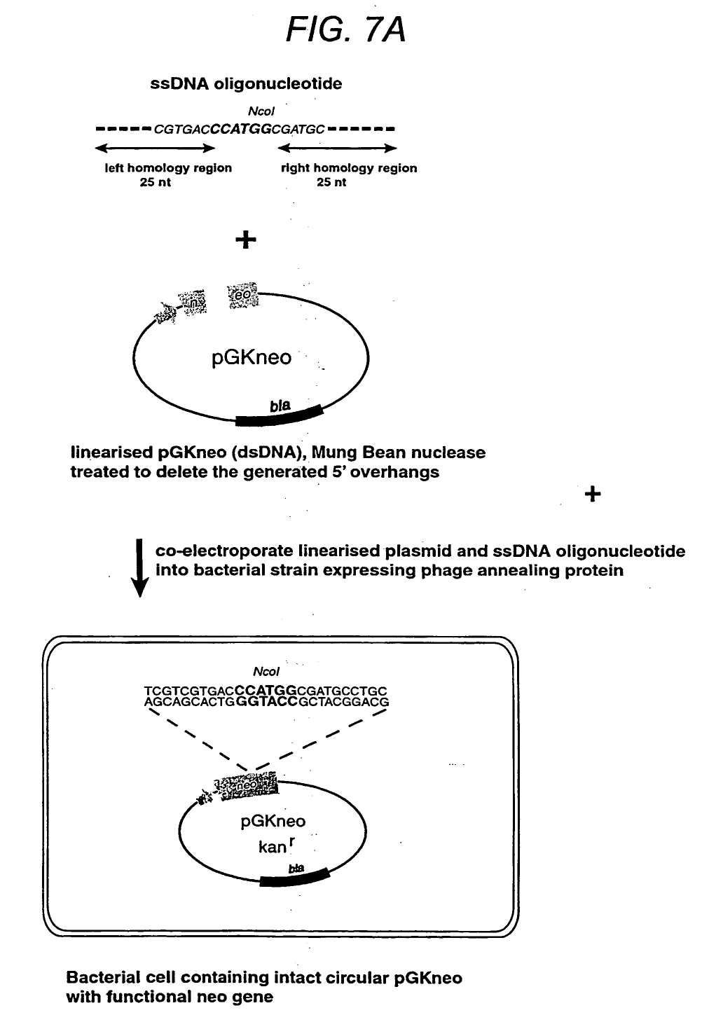

The described recombination activity of phage annealing proteins can also be applied to recircularise linearised plasmids, in this example to include sequence previously present between the homology regions of the ssDNA oligonucleotide, as is shown in FIG. 7. Here, a linearised plasmid was co-electroporated with a ssDNA oligonucleotide into a host strain that expressed a phage annealing protein. Selection pressure was exerted only for expression of the selectable marker gene bla present on the linearised plasmid. Thus, no selection pressure was applied for the region that recombines. After recombination, intact circular plasmids were obtained which contained the sequence originally present between the homology regions of the ssDNA oligonucleotide.

The detailed experimental procedures for this experiment are as follows. NcoI linearised pGKneo (NcoI has a unique recognition site in the neo gene of pGKneo, see experimental protocol to FIG. 1) was mung bean nuclease treated according to the manufacturer's instructions (New England Biolabs). Mung bean nuclease treatment removes the 5′overhangs generated by NcoI and deletes 4 nucleotides from the neo gene of pGKneo. These four nucleotides are present on the oligonucleotide, and through repair recombination a functional neo gene can thus be restored. The indicated phage proteins were inducibly expressed in JC5519 from pBAD24-based plasmids containing the respective genes. Electrocompetent cells which inducibly express the indicated proteins were prepared exactly as described above, as were electroporation, selection and normalisation of the obtained recombination efficiencies. For ampicillin selection, a concentration of 50 μg/ml was used.

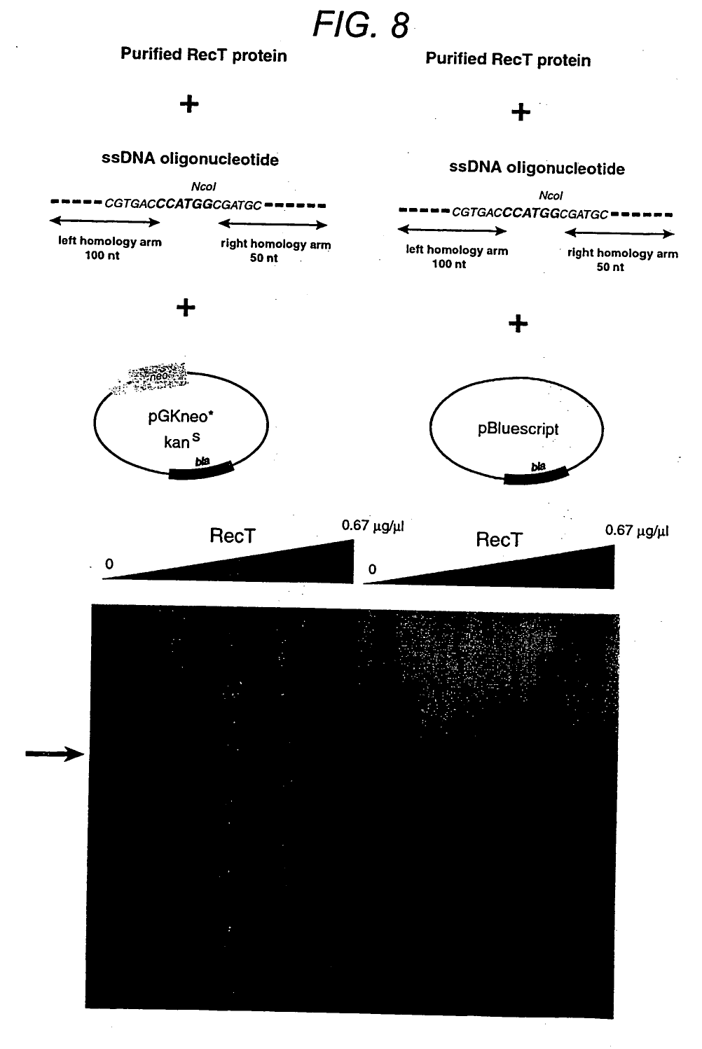

Although the exact mechanism by which the described activity functions is not precisely known, phage annealing proteins are known to be capable of binding efficiently to ssDNA and/or dsDNA molecules (RecT, see Noirot and Kolodner, J Biol Chem 273 (1998), 12274-12280 and references therein; Redβ, see Muniyappa and Radding, J Biol Chem 261 (1986), 7472-7478; Karakousis et al., J Mol Biol 276 (1998), 721-731; Li et al., J Mol Biol 276 (1998), 733-744 and references therein). Moreover, the phage annealing protein RecT can form an in vitro complex between itself, a ssDNA oligonucleotide and an intact circular plasmid. Such complexes lead to the formation of joint molecules (a joint molecule which consisted of the plasmid and the ssDNA oligonucleotide) which were found to be stable after removal of RecT. However, the formation of stable joint molecules was found to be dependent on shared homology regions between the ssDNA oligonucleotide and the plasmid (FIG. 8).

For this experiment, the following procedure was used. The indicated ssDNA oligonucleotide was end-labeled according to the manufacturer instructions (New England Biolabs). RecT was purified to homogeneity from a bacterial strain overexpressing RecT, as described before (Hall et al., J Bacteriol 175 (1993), 277-287). RecT was incubated at the indicated concentration range for 20 minutes at 25 C with 3 μg of the labeled ssDNA oligonucleotide in a buffer consisting of 25 mM NaCl, 20 mM TrisHCl pH=7.5, 100 μg/ml BSA, 0.5 mM DTT, in a total volume of 27 μl. Subsequently, 3 μl (corresponding to 2 μg) of a plasmid was added and incubation was continued at 37 C for an additional 45 minutes. The samples were deproteinised by addition of concentration of proteinase K to a final concentration of 0.5 mg/ml, SDS to a final concentration of 0.1% and EDTA pH=8 to a final concentration of 50 mM and incubation at 37 C for 10 minutes. 20 μl of a sample was loaded onto a 0.8% agarose gel followed by electrophoresis at 75 V for 2 hours. Signals were detected after blotting to nitrocellulose and exposing to photographic film.

Such in vitro made joint molecules may be usable to mediate recombination directly in a host cell that does not need to express any phage annealing protein. Also, use of in vitro-formed joint molecules will increase the efficiency of the described activity in any application. Also, DNA molecules (for example ssDNA oligonucleotides) that are coated by a phage annealing protein should recombine with higher efficiency compared to a ‘naked’ DNA molecule.

The described activity of phage annealing proteins can be applied for the engineering of several molecules of various type and conformation. An overview is given in Table 5.

Table 5. Overview of the types of first and second DNA molecules that can be used for engineering by the listed phage annealing proteins. Recombination reactions constitute the recombination between a first molecule and a second molecule, mediated by a phage annealing protein. Several examples can be found in the data given in the previous figures and tables. Other examples, including the use of annealed DNA oligonucleotides and RNA molecules are only listed here.

### Example 9

Repair by Homologous Recombination in ES Cells

Plasmids

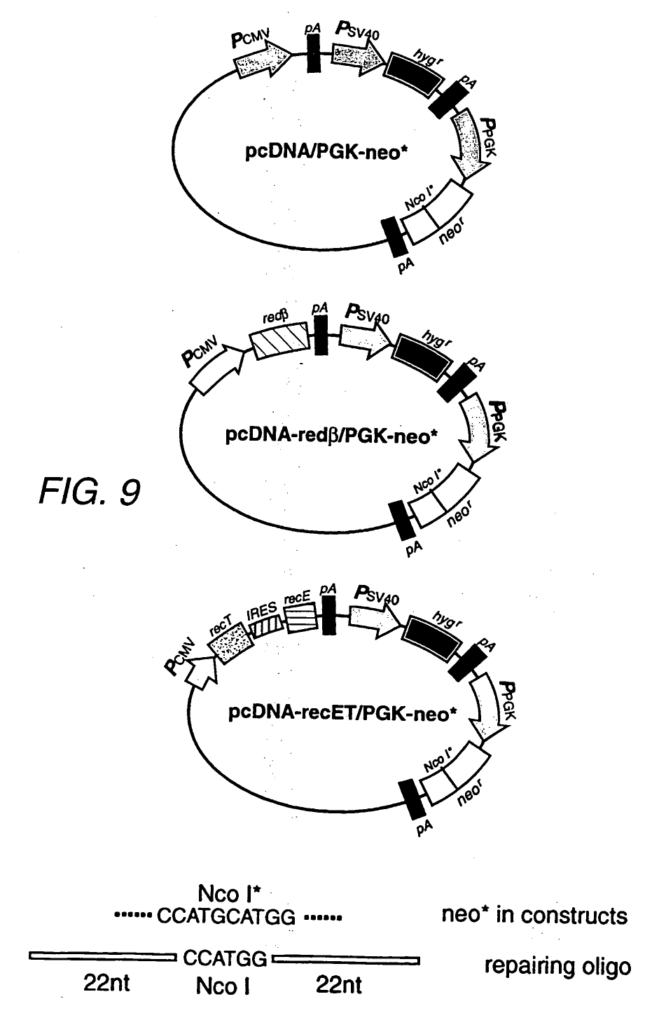

A DNA fragment which consists of the PKG promoter and neo* was inserted into the Bst1 107 I site of pcDNA3/hyg(−) (Invitrogen) to generate pcDNA /PGK-neo*. The redβ gene and the recE/IRES/recT fragment were inserted under the CMV promoter in pcDNA/PGK-neo* to generate pcDNA-redβ/PGK-neo* and pcDNA-recET/PGK-neo* (see FIG. 9).

Repair Oligonucleotide

A 50 nucleotide (nt) oligonucleotide was synthesized according to the sequence of neo gene in the region of Nco I site. This oligonucleotide consists of two 22 nt homology regions, each flanking the correct Nco I sequence (see; FIG. 9 bottom). The sequence of this oligonucleotide is as follows:

5′ ACGGCGAGGATCTCGTCGTGACCCATGGCGATGCCTGCTTGCCGAATATC3′

Mouse ES Cells

Mouse ES cells were cultured in DMEM (Gibco & BRL) with 4% glucose (Gibco & BRL), 15% FCS (PAA), 100 μg/ml of penicillin/streptomycin (Gibco & BRL), 100 μM of Non-Essential Amino Acids (Seromed), 1 mM of Sodium Pyruvate(Gibco & BRL), 1 μM of Beta-mercaptoethanol (Sigma), 2 mM of L-Glutamine (Gibco & BRL) and 500 U/ml of LIF “ESGROTM” (Gibco & BRL).

Plasmid DNA

Expression plasmids (FIG. 1) were isolated using a Qiagen Maxi-prep kit and digested with Ahd I (New England Biolabs) to generate linear DNA. After precipitation, DNA was resuspended in PBS (Gibco & BRL) at 0.5 mg/ml.

Electroporation

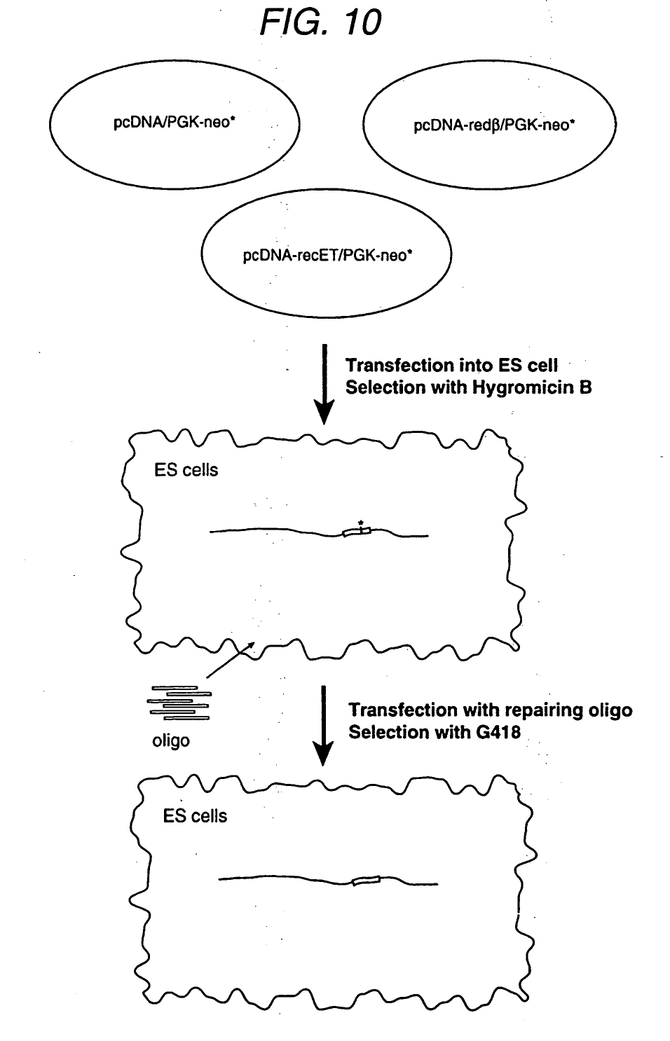

ES cells on a 10-cm dish were rinsed once with 10 ml of PBS after they were confluent. 1 ml of trypsin/EDTA (Gibco & BRL) solution was added to the dish. The dish was incubated in the incubator for 3-5 minutes. 10 ml of ES culture medium were added into the dish and ES cells were separated into single cell by pipetting up and down. ES cells were spun down at 1,000 rpm for 5 minutes. The supernatant was removed and the cell pellet was resuspended in 0.8 ml of PBS. 20 μg of plasmid DNA or 5 μg of the oligonucleotide were mixed with ES cells and placed into a 4-mm electroporation cuvette. The mixture of DNA plus ES cells was electroporated at 240 v. The electroporated cells were transferred into a gelatin-coated dish and 10 ml of culture medium was added (see FIG. 10).

Selection

The medium of the transfected cells was changed every day and selection antibiotic was added 48 hours post transfection. Colonies were seen after around 10 days of selection. The concentration of antibiotics used were:

G418—200 μg/ml (Gibco & BRL)

Hygromycin B—400 μg/ml (Boehringer Mannheim)

Experimental Procedure

1, ES cells were cultured and transfected with expression plasmids (pcDNA/PGK-neo*, pcDNA-redβ/PGK-neo* and pcDNA-recET/PGK-neo*, thus 3 separate transfections were carried out).

2, After selection with Hygromycin B for 10 days, 12 colonies were picked from each dish and transferred into 24-well plates.

3, Cells were transferred into 6-well plates after they were confluent in 24-well plates.

4, Cells were transferred into 10-cm dish after they were confluent in 6-well plates.

5, Transfection with the oligonucleotide was performed after the cells were 70-80% confluent.

6, Transfected cells were selected by G418 and Hygromycin.

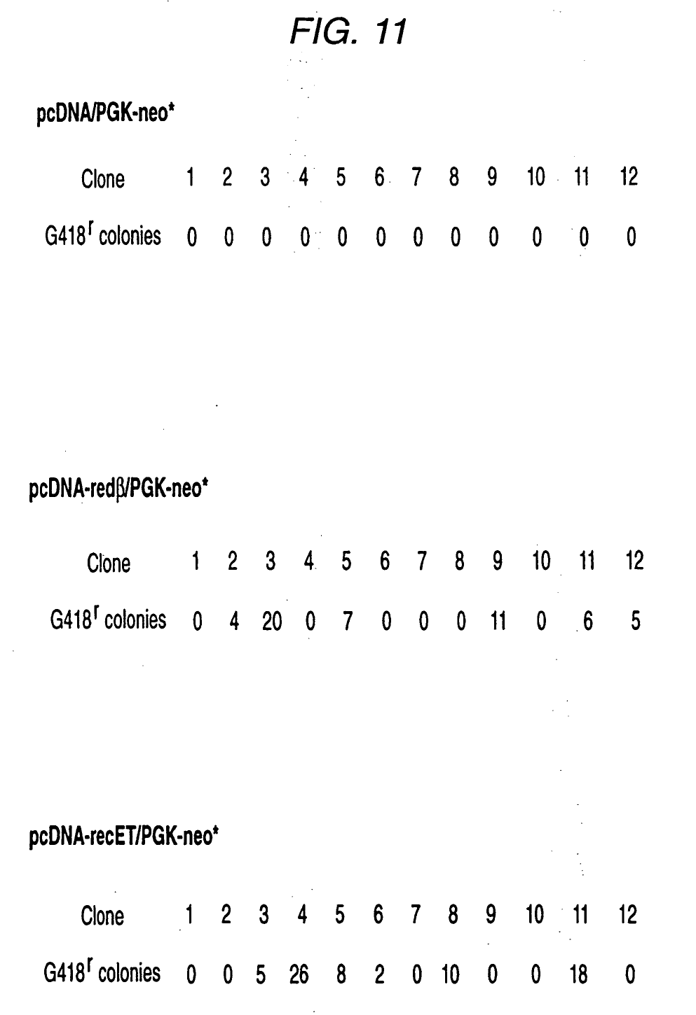

7, Colonies were counted (FIG. 11).

It can be seen from the results presented in FIG. 11 that the oligonucleotide successfully repaired the mutated neo* gene in ES cells that were cultured and transfected with the expression plasmids pcDNA-redβ/PGK-neo* and pcDNA-recET/PGK-neo*, as compared to control cells transfected with pcDNA/PGK-neo*.

### Example 10

Introduction of Short Fragments into Chosen sites in BACs

The recombination method of the present invention can also be performed on bacterial artificial chromosomes (BACs), which have become the premier cloning vector due to their large capacity for length of insertions.

The BAC used in this example contains the mouse M22 gene and is over 150 kb in size. In order to create a substrate that facilitated the evaluation of the ss oligonucleotide recombination step, the Mll BAC was first subjected to a round of ET recombination (WO99/29837) to place a cassette containing the Tn5 kanamycin resistance gene (neo) and streptomycin counterselection gene (rpsL) into a predetermined site. This was accomplished by transforming E.coli containing the Mll BAC with the pSC101/BAD/γβα expression vector. Arabinose was added during culture of the cells to induce expression of the phage recombination proteins γβα followed by preparation of electrocompetent cells. These electrocompetent cells were electroporated with a linear PCR fragment that had been generated using two 60 nucleotide oligonucleotides which contained, at their 5′ ends, 40 nucleotides of sequence identical to chosen regions in the BAC, and at their 3′ ends, 20 nucleotides of sequence that serve as primers on the rpsL/neo template for the PCR reaction. Integration of the PCR fragment into the BAC by ET cloning was identified by selection for kanamycin resistance.

Of more than 5,000 colonies that were Kanamycin (15 μg/ml) resistant, 22 colonies were analysed with restriction digestion and all were correct. To check that the rpsL gene was functional, these 22 were also streaked onto Streptomycin plates (50 μg/ml). 20 of these clones were sensitive to Streptomycin and 4 were taken for the next step.

Ss oligonucleotide recombination was used to insert a short sequence, here either an Xho1 restriction site or the 34 bp FRT (FLP recombination target), into the BAC. Two single-stranded oligos were used to delete the rpsL-neo cassette. Both oligos had 25 nucleotides (nt) homology to the Mll BAC sequence immediately adjacent to the insertion site of the rpsL/neo cassette. In the middle of the oligos, an Xho I site (oligonucleotide 1) or an FRT (oligonucleotide 2), were included. After preparation of ET electro-competent cells (as before), the oligos were electroporated and ss oligonucleotide recombination was selected by plating on Streptomycin (50 μg/ml) to select for the loss of the rpsL gene.

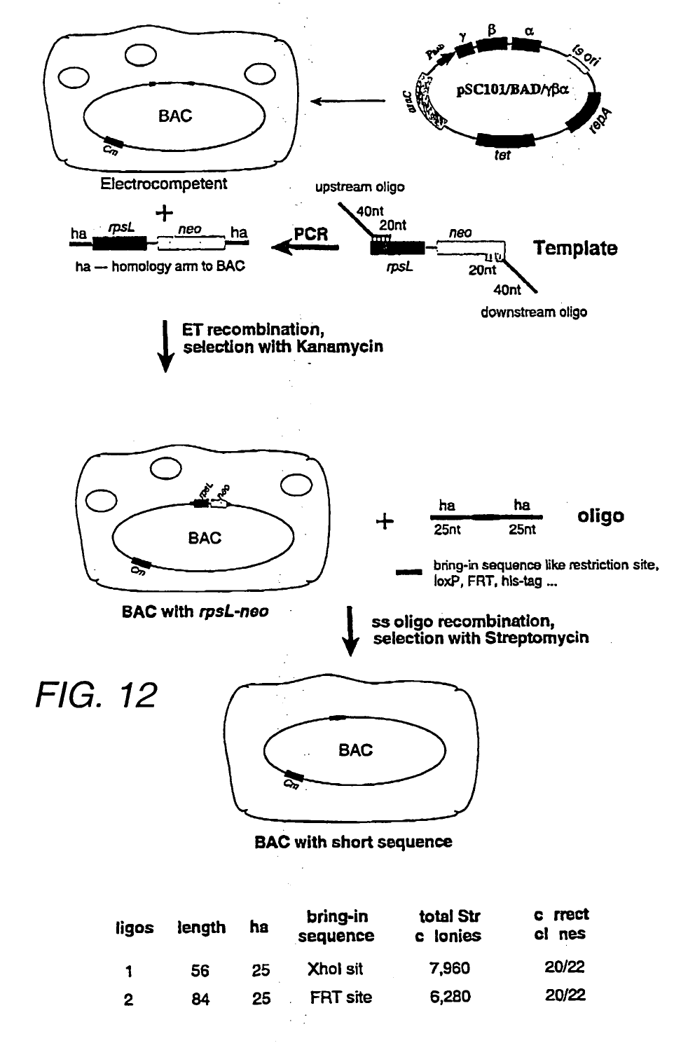

Colonies that grew on the plates were counted (shown at the bottom of the FIG. 12). 22 colonies were picked and BAC DNA was analysed by restriction digestion. In both cases 20 out of 22 were correct.

This Experiment Shows that ss Oligonucleotide Recombination—

a. can delete a region from a BAC (here the rpsL/neo cassette);

b. can be used to introduce new sequence into a specific site. Here the sequences were short since short sequence regions, up to 100 nts in length, can be easily included during oligonucleotide synthesis. However longer sequences can be included if the ss DNA is prepared by other methods from longer DNA sources.

c. is simple, robust and efficient.

Experimental Procedure for Modification of BAC by ss Oligonucleotide Recombination Using pSC100BAD/βγα Expression System

Transform pSC101/βγαA expression plasmids into mouse MLL BAC host cells (HS996), as described in the conventional transformation method. Use a cooling Eppendorf centrifuge with the temperature set at 2° C. to cool the cells. Before the experiment, cool down the dH₂O for at least 3 hours on ice or take cooled dH₂O from the fridge and put it on ice. Electroporation cuvettes should also be put on ice.

1. Inoculate a single colony containing Mll BAC with the rpsL-neo cassette plus pSC101/BAD/γβα in 1.4 ml LB medium with tetracyclin (5 μg/ml), kanamycin (15 μg/ml) and chloramphenicol (15 μg/ml) in an Eppendorf tube having a hole in the lid. Incubate the tube in a heating block at 30° C. with shaking, for 4-5 hours till OD600˜0.15-0.2.

or

Add 30 μl of overnight culture in 1.4 ml LB medium with tetracyclin (5 μg/ml), kanamycin (15 μg/ml) and chloramphenicol (15 μg/ml) in an Eppendorf tube having a hole in the lid. Incubate the tube in a heating block at 30° C. with shaking, for about 2 hours till OD600˜0.2.

then

2. Add L-arabinose to 0.1-0.2% (final) to induce the expression of recombinases.

3. Transfer into a 37° C. heating block and incubate at 37° C. for 45-60 minutes until OD600˜0.35-0.4.

4. Spin down the cells using the highest speed for 30 seconds in an Eppendorf centrifuge at room temperature.

5. Discard the supernatant and put the tube on ice.

6. Resuspend the cells in 1.0 ml of ice-cooled dH₂O or 10% ice-cooled glycerol on ice.

7. Spin down the cells using the highest speed-for 30 seconds and discard the supernatant.

8. Resuspend the cells in 1.0 ml of ice-cooled dH₂O or 10% ice-cooled glycerol on ice again.

9. Spin down the cells using the highest speed for 30 seconds.

10. Discard the supernatant by using 1 ml pipette and leave around 20-30 μl of solution.

11. Add 1 μl of oligonucleotide (50 μM) in dH₂O and remove into an ice-cooled electroporator cuvette (1 mm).

12. Electroporate the cells at 1,350 V using an Eppendorf electroporator.

13. Add 1 ml of LB medium and incubate at 37° C. for 75 minutes.

14. Transfer the cells on plates with chloramphenicol (15 μg/ml) and streptomycin (15 μg/ml) or other antibiotics.

15. Incubate the plates at 37° C. overnight. The ET plasmid (pSC101/BAD/γβα) will be lost at 37° C.

### Conclusion of Example 10

BACs have often been shown to be the most demanding templates for modification. By demonstrating that the recombination techniques of the present invention can work with BACs, all other templates in E. coli, including the E. coli chromosome, PACs and other low copy templates, as well as medium and high copy plasmids, can also be modified by ss oligonucleotide recombination.

### Example 11

High-Throuph-Put Sequence Deletion and Introduction of Short Fragments in a Mouse Mll BAC by Single-Stranded Oligonucleotide via ET Recombination.

This example further demonstrates the ability of the recombination technique of the present invention to work with BACs.

A BAC was modified by a first round of ET recombination, as in Example 10. However, in this case, the BAC already contained the Tn5 kanamycin resistance gene (neo), which had been introduced in a previous round of ET recombination (not shown). The neo gene itself was disrupted by introduction of a lacZ/Zeo cassette by selection for acquisition of zeocin resistance after ET recombination. In this experiment, the ColE1 origin plasmid pBAD αβγ was used, rather than the pSC101 plasmid used in Example 10.

The lacZ/zeo cassette is around 3.45 kb long and consists of the lacZ gene fused with the zeocin resistant gene (zeo) at the 3′ end. After integration into the neo gene in the modified Mll BAC by ET recombination, plating of an aliquot to assay for zeocin resistance. (10 μg/ml) indicated that more than 10⁵ colonies were correct recombinants. 50 colonies were then streaked on kanamycin plates to evaluate the loss of kanamycin resistance, all of which were sensitive to kanamycin, indicating correct recombination. 22 colonies were further analysed by restriction digestion and all were correct. 4 correct clones were used for the next step.

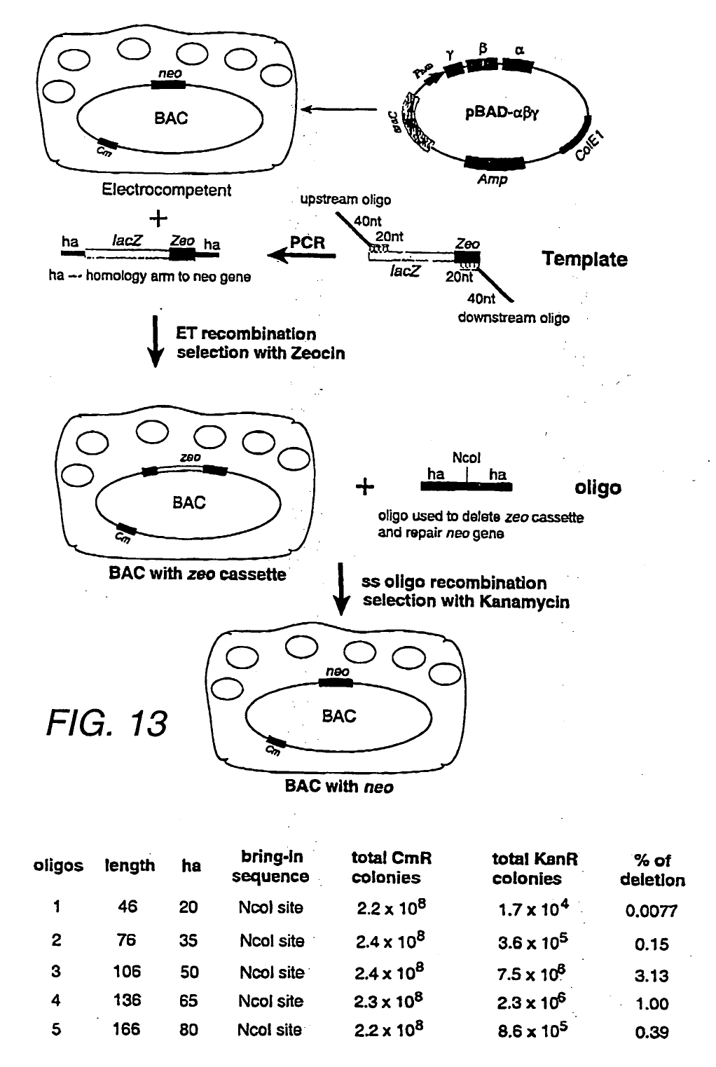

A set of 5 ss oligonucleotides were synthesised which differed in the length of the sequence identical to each side of the disruption point of the neo gene. The oligonucleotides had either 20, 35, 50, 65 or 80 nucleotides of sequence identity in their homology arms (ha) either side of a 6 nucleotide Nco1 restriction site. Each oligonucleotide, if correctly recombined into the BAC by ss oligonucleotide recombination, will reconstitute the neo gene with a Nco1 site and delete the lacZ/zeo cassette. Hence, correct recombination can be scored by acquisition of kanamycin resistance. After preparation of ET electro-competent cells (see experimental procedure below), and electroporation of the oligonucleotides, the cells were diluted and plated on kanamycin (15 μg/ml) plus chloramphenicol (15 μg/ml) plates or on chloramphenicol (15 μg/ml) only plates and the number of colonies was scored. The results are shown at the bottom of FIG. 13. 22 kanamycin-resistant colonies were analysed by Nco I restriction digestion and all were correct.

Experimental Procedure for Modification of BAC with an Oligonucleotide Using the pBAD/γβα Expression System

Transform the pBAD/βγα expression plasmid into mouse Mll BAC host cells (HS996) as described in the conventional transformation method. Use a cooling centrifuge to cool down the E.coli cells, setting the temperature at −5° C. Before the experiment, cool down dH₂O for at least 3 hours on ice or take cooled dH₂O from the fridge and put it on ice. The eletroporation cuvettes should also be put on ice.

1. Inoculate a single colony containing Mll BAC with the neo-lacZ-zeo cassette plus pBAD-γβα in 1.4 ml LB medium with ampicillin (100 μg/ml), zeocin (10 μg/ml) and chloramphenicol (15 μg/ml) in an Eppendorf tube having a hole in the lid.

2. Incubate the tube in a heating block at 37° C. with shaking overnight.

3. Add 0.3 ml of overnight culture in 30 ml LB medium with ampicillin (100 μg/ml), zeocin (10 μg/ml) and chloramphenicol (15 μg/ml) in a 200 ml-flask.

4. Incubate the flask at 37° C. with shaking, for about 2 hours till OD600˜0.2.

5. Add L-arabinose to 0. 1-0.2% (final) to induce the expression of recombinases.

6. Incubate at 37° C. for 45-60 minutes until OD600˜0.35-0.4.

7. Transfer the culture in a centrifuge tube and spin down the cells at 7,000 rpm for 6 minutes at −50° C.

8. Discard the supernatant and put the tube on ice.

9. Resuspend the cells in 30 ml of ice-cooled dH₂O or 10% ice-cooled glycerol on ice.

10. Spin down the cells at 7,000 rpm for 6 minutes at −5° C. and discard the supernatant.

11. Resuspend the cells in 30 ml of ice-cooled dH₂O or 10% ice-cooled glycerol on ice again.

12. Spin down the cells at 7,000 rpm for 6 minutes at −5° C.

13. Discard the supernatant and immediately clean up the tube by using tissue, leaving around 20-30 μl of solution.

14. Transfer the competent cells into an eppendorf tube and add 1 μl of oligonucleotide (50 μM) in dH₂O.

15. Remove the mixture into an ice-cooled electroporator cuvette (1 mm) and electroporate the cells at 1,350 V using an Eppendorf electroporator.

16. Add 1 ml of LB medium and incubate at 37° C. for 75 minutes.

17. Transfer the cells onto plates with chloramphenicol (15 μg/ml) and kanamycin (15 μg/ml).

18. Incubate the plates at 37° C. overnight.

### Conclusion to Example 11

In addition to establishing the same conclusions as Example 10 by using a different strategy and a different expression plasmid, this experiment measures the absolute efficiency of ss oligonucleotide recombination. At an apparent optimum length of 50 nucleotides in the homology arms, 3% of the total number of colonies were correctly recombined. Given this remarkable efficiency, it is apparent that selection for antibiotic resistance is not required and that simple physical methodologies, such as restriction analysis, PCR, colony PCR or colony hybridization, can be used to identify the correct recombinants.

Discussion

We here describe a novel recombination activity, in which a single phage annealing protein mediates recombination of two molecules by repair recombination through shared homologous sequences. Using this activity, several engineering strategies such as the deletion (FIG. 1A), insertion (from nucleotides and short operational sequences such as protein tags (FIG. 1B) to several kilobasepairs) or substitution of sequences are feasible to a range of molecules. Some key characteristics of this activity are summarised below. Many of these characteristics distinguish the activity described here from ET recombination (see above), which also requires the expression of a phage annealing protein. Where relevant, these differences have been emphasised.

The described activity allows the deletion, insertion and substitution of one or many nucleotides in a range of molecules

By design of the sequences of the homology regions, the described activity can be applied to DNA engineering of molecules at any modifiable position desired.

The described activity is feasible for modification of exogenous (for example plasmids) and endogenous (chromosomal) DNA.

The described activity requires the expression of a phage annealing protein. However, expression of the orthologous exonuclease partner is not required. This is in contrast with ET recombination, in which the expression of the annealing protein and its orthologous exonuclease partner are strictly required (Muyrers et al., Genes Dev 14 (2000), 1971-1982). Furthermore, other recombination pathways in E. coli were found to be incapable of mediating the described activity.

The required shared homology length needed is very short (shorter than for ET recombination, Zhang et al., Nature Genet 20 (1998), 123-128; Muyrers et al., Genes Dev 14 (2000), 1971-1982)

Until the lengths tested so far, recombination efficiency continues to increase with increasing shared homology region length

In vitro, phage annealing proteins can coat molecules and are capable of forming joint molecules between homology region sharing molecules

In the described activity, point mutations in the homology regions are not introduced into the recombinant (FIG. 4).

In the mechanism by which the described activity functions, it is unlikely that the second molecule (Table 5) only functions as a primer for replication or an Okazaki fragment, since second molecules containing dideoxy residues can be used for the described activity (FIG. 5) and because plasmid and chromosomal engineering can be done using single-stranded molecules from both leading and lagging strands (Table 3, FIG. 6)

RecBCD expression does not inhibit the described activity, whereas it inhibits ET recombination (Zhang et al., Nature Genet 20 (1998), 123-128).

The P22 recombination system constituting only Erf, or any combination of Arf, Erf, Abc1 and Abc2 is proficient for the described activity, but not for ET recombination (probably due to the absence of an orthologous exonuclease partner for Erf).

In the described activity, an increased amount of nucleotides that need to recombine from the oligonucleotide into the circular plasmid correlates with a decrease in recombination efficiency. No such effect is seen for ET recombination.

RNA molecules can be used in the described activity.

The described activity allows a widely applicable strategy for DNA engineering. Given the high efficiency of the described activity, selection methods to identify the correct recombinants from the total pool of electroporated cells that are not based on some form of antibiotic selection are feasible. Such selection methods include, for example, selective PCR methods, restriction enzyme analysis and colony hybridisation. Furthermore, the stability of target molecules is not endangered by the presence of a functional homologous recombination pathway which requires the presence of the orthologous exonuclease partner protein in addition to a phage annealing protein. The efficiency of DNA engineering using the described activity can be increased further by using coated or joint molecules in the recombination strategy (see FIG. 8).

The described activity may also be useful for genetic manipulation in other species or cells, which are capable of expressing a phage annealing protein from an endogenous or exogenous source. Alternatively, in vitro pre-made joint or coated molecules (this method is described in FIG. 8 and can be applied to any type of joint or coated molecule, using any first and/or second DNA molecule and any annealing protein listed in Table 5) can be used for repair and homologous recombination in any species or cells that do not need to express any phage annealing protein and still allow the described activity.

The findings, detailed in Example 9, that RecT as well as Redβ allow the targeted modification of a locus present on an ES-cell chromosome, by using a DNA molecule (here: an oligonucleotide) which shares only very short homology regions to said chromosomal locus, is of high importance.

First, it demonstrates that Redβ and RecT (and by inference, RecT and RecE) are functional in eukaryotic cells, such as ES cells. Therefore, it is likely that these proteins, as well as their functional homologues, function in other eukaryotic cells and organisms as well.

Second, this opens the door to new possibilities in DNA engineering of eukaryotic cells and organisms. Until now, for example plant cell lines, fly cell lines etc., and most of the somatic cell lines of mammals have very low (if any) efficiency of homologous recombination. It is therefore very difficult at present to target a specific locus in such cells; the same is true for targeted modification of most eukaryotic organisms. This can be greatly simplified by using phage annealing proteins (or their functional homologues) to modify a chosen locus using a targeting molecule (such as an oligonucleotide, however other nucleic acid molecules may be used) with very short homology regions. The modified locus can be present endogenously (on a chromosome), or exogenously.

Third, until now, in most homologous recombination-proficient eukaryotic cells (however, not in yeast), targeting molecules with a long homology region length, which needed to be constructed into a dedicated targeting construct, were needed. Using the method we describe here, no targeting construct needs to be constructed; instead a synthetic oligonucleotide is purchased and directly used. Also, the overall targeted modification efficiency may be increased significantly by application of this finding.

Fourth, this finding opens access to DNA modification in higher eukaryotes without applying selection markers. From the experiment presented in Example 9, it is evident that in a part of the colonies analyzed, targeted modification had taken place (N.B. this modification was detected by repair of a selectable marker, however this was only done after the modification had already taken place).

Fifth, the finding that RecT and Redβ are functional in ES cells confirms that joint and coated molecules are functional as well. Thus, by pre-incubation with RecT or Redβ in vitro, the efficiency of targeted modification in eukaryotic cells and animals is likely to be increased. By using joint and coated molecules, it is also likely that targeted modification is feasible in cells and organisms with low (or no) inherent potential for homologous recombination.

Sixth, ET recombination (described in co-owned patent applications WO9929837 and WO0104288) can potentially be applied directly in higher eukaryotes as well. Although ET recombination strictly depends on both components (RecE and RecT, or Redα and Redβ; see Muyrers et al. Genes Dev 14, 1971-1982 (2000)), the finding that at least RecT and Redβ are functional is encouraging, and implies that ET recombination could be developed in higher eukaryotes directly.

Thus, this finding and its implications (such as use of coated molecules, joint molecules, or eukaryotic ET recombination) can be used to simplify and increase the efficiency of targeted modification of eukaryotic cells and organisms which are already proficient in homologous recombination, and may enable targeted modification in eukaryotic cells and organisms which are not proficient in homologous recombination. RecT or Redβ (or any of their functional homologues; if required other components can also be included, such as RecE, or Redα, RecA, their functional homologues, etc) can thus be provided in vivo and/or in vitro (joint and coated molecules).

In all, the significance of this finding is potentially similar in impact to the use of Cre (a bacterial protein) in higher eukaryotic cells, and opens access to a novel logic for DNA engineering in eukaryotes.

TABLE 1
TABLES

| | | Recombination | Normalised |
|---|---|---|---|
| | | mediating pathway | recombination |
| Strain | Genotype | or protein(s) | efficiency |
| MM294 | wild-type | RccA/RccBCD | 0 |
| MM294 − pBADET | wild-type | RecA/RecBCD; RecE/RecT | 50 |
| MM294 + pBADETγ | wild-type | RecA, no RecBCD; RecE/RecT | 46 |
| MM294 \| pBADαβ | wild-type | RecA/RecBCD; Redα/Redβ | 55 |
| MM294 + pBADαβγ | wild-type | RccA, no RccBCD; Rcdα Rcdβ | 52 |
| JC8679 | sbcA, rccBC | RecE/RecT | 45 |
| JC13031 | rac, recJ | RecA/RecBCD | 0 |
| JM103 | sbcBC | RecA/RecBCD; RecF-pathway | 0.1 |
| JC9387 | sbcBC, rac, recBC | RecF-pathway | 0.2 |
| JC15329 | sbcBC, recA, recBC, rac | RecF-pathway? | 0 |
| JC8111 | sbcBC, rac, recBC, recF | RecF-pathway? | 0.1 |
| JC5519 | recBC | RecA, no RecBCD | 0 |
| JC5519 + pBADrccT | recBC | RecA, no RecBCD; RecT | 51 |
| JC5519 + pBADrecET | recBC | RecA, no RecBCD; RecE/RecT | 49 |
| JC5519 + pBADredβ | recBC | RecA, no RecBCD; Redβ | 51 |
| JC5519 \| pBADredαβ | recBC | RecA, no RecBCD; Redα/Redβ | 52 |
| JC5519 + pBADerf | recBC | RecA, no RecBCD; Erf | 35 |
| JC5519 + pBADarferf | recBC | RecA, no RecBCD; Arf/Erf | 37 |
| JC9366 | recA | — | 0 |
| JC9366 + pBADrccT | recA | RecT | 47 |
| JC9366 + pBADrecET | recA | RecE/RecT | 45 |
| JC9366 + pBADrecETγ | recA | RecE/RecT/Redγ | 50 |
| JC9366 \| pBADredβ | recA | Redβ | 45 |
| JC9366 + pBADredαβ | rccA | Redα/Redβ | 54 |
| JC9366 + pBADredαβγ | recA | Redα/Redβ/Redγ | 51 |
| JC9366 + pBADerf | recA | Erf | 32 |
| JC9366 + pBADarferf | recA | Arf/Erf | 38 |
| JC9366 + pBADarferfabc1 | recA | Arf/Erf Abc1 | 30 |
| JC9366 + pBADarferfabc1abc2 | recA | Arf/Erf/Abc1/Abc2 | 34 |

TABLE 2

| ssDNA oligonucleotide | | Normalised recombination | |
|---|---|---|---|
| 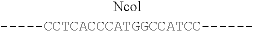 | | efficiency, mediated by: | |
| 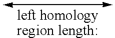 | 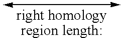 | RecT | Redβ |
| 0 | 8 | 0 | 0 |
| 0 | 10 | 0 | 0 |
| 0 | 15 | 0 | 0 |
| 0 | 20 | 0 | 0 |
| 0 | 100 | 0 | 0 |
| 5 | 0 | 0 | 0 |
| 10 | 0 | 0 | 0 |
| 15 | 0 | 0 | 0 |
| 20 | 0 | 0 | 0 |
| 100 | 0 | 0 | 0 |
| 20 | 20 | 28 | 35 |

TABLE 3

| | Normalised recombinatlon | |
|---|---|---|
| | mediated by: | |
| ssDNA oligonucleotide efficiency | RecT | Redβ |
| complementary to bottom strand: | | |
| 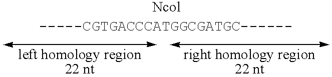 | 46 | 51 |
| 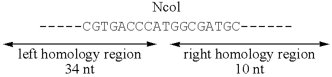 | 35 | 33 |
| 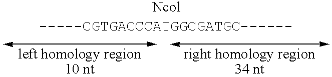 | 19 | 22 |
| complementary to top strand: | | |
| 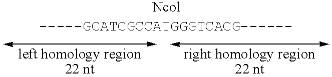 | 22 | 24 |
| 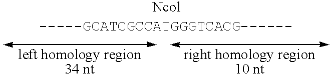 | 18 | 19 |
| 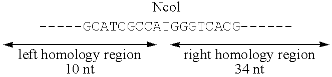 | 9 | 11 |

TABLE 4

| | Colony | Colony number |
|---|---|---|
| Annealing protein | number on LB-amp plates | on LB-amp/kan plates |
| RecT | 10⁷ | 5 × 10⁴ |
| Redβ | 10⁷ | 7 × 10⁴ |

TABLE 5

| | | | Phage annealing |
|---|---|---|---|
| First molecule | Second molecule | | protein |
| intact dsDNA circular molecule | ssDNA (both orientation) | 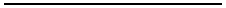 | RecT |
| linoarised dsDNA molecule | annealed oligo's, no overhang | 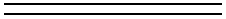 | Redβ |
| E. coli chromosome | annealed oligo's, 5 overhang | 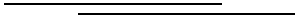 | Ert |
| | annealed oligo's, 3 overhang ssRNA (both orientations) | 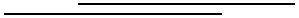 | |

1. A method for altering the sequence of a nucleic acid molecule, said method comprising the steps of:
a) bringing a first nucleic acid molecule into contact with a second nucleic acid molecule in the presence of a phage annealing protein, or a functional equivalent or fragment thereof, wherein said first nucleic acid molecule comprises at least two regions of shared sequence homology with the second nucleic acid molecule, under conditions suitable for repair recombination to occur between said first and second nucleic acid molecules; and
b) selecting a nucleic acid molecule whose sequence has been altered so as to include sequence from said second nucleic acid molecule.

2. A method according to claim 1, with the proviso that the RecE/Redα protein is not present during any sequence alteration reaction that is carried out in a prokaryotic cell.

3. A method according to claim 1 or claim 2, wherein said phage annealing protein is contained within or encoded by a host species.

4. A method according to claim 3, wherein said host species is a virus, a parasite, a prokaryote or a eukaryote cell.

5. The method according to claim 4, wherein the host species is a gram-negative bacterial cell.

6. The method according to claim 5, wherein the bacterial cell is an Escherichia coli cell.

7. The method according to claim 6, wherein the Escherichia coli cell is a cell of an Escherichia coli K12 strain, such as a JC5519, JC8679 or JC9604 strain.

8. The method according to claim 4, wherein the host species is an ES cell.

9. The method according to claim 8, wherein the host species is a mouse ES cell.

10. The method according to any one of claims 3-9, wherein the host species is transformed with at least one vector capable of expressing a gene encoding a phage annealing protein, functional equivalent or fragment thereof.

11. The method of claim 10, wherein the expression of the gene encoding the phage annealing protein, functional equivalent or fragment is under control of a regulatable promoter.

12. A method according to any one of claims 3-9, wherein the phage annealing protein, functional equivalent or fragment is expressed from a messenger RNA molecule that is introduced into the host species.

13. A method according to any one of the preceding claims, wherein said phage annealing protein is selected from the group consisting of RecT (rac prophage), Redβ (phage λ), and Erf (p22), or a functional equivalent or active fragment thereof.

14. A method according to any one of the preceding claims, wherein the first nucleic acid molecule is linear.

15. A method according to claim 14, wherein said first nucleic acid molecule is selected from the group consisting of a single-stranded DNA molecule, a single-stranded RNA molecule, a double-stranded DNA molecule, a double-stranded DNA molecule with 5′ overhang, and a double-stranded DNA molecule with 3′ overhang.

16. A method according to claim 15, wherein said first nucleic acid molecule is a single-stranded nucleic acid molecule.

17. The method according to any one of claims 14-16, wherein the first DNA molecule is obtained by an amplification reaction.

18. The method according to any one of the preceding claims, wherein the second nucleic acid molecule is circular.

19. The method according to claim 18, wherein the second nucleic acid molecule is an extrachromosomal nucleic acid molecule containing an origin of replication which is operative in a host cell.

20. The method according to claim 18 or claim 19, wherein the second nucleic acid molecule is selected from the group consisting of plasmids, cosmids, P1 vectors, BAC vectors and PAC vectors.

21. The method according to any one of claims 18-20, wherein the second nucleic acid molecule is a host cell chromosome.

22. The method according to any one of the previous claims wherein the regions of sequence homology shared between said first nucleic acid molecule and said second nucleic acid molecule are at least 9 nucleotides each.

23. The method according to any one of claims 3-22, wherein the first and/or second nucleic acid molecules are introduced into the host species by transformation.

24. The method according to claim 23, wherein the transformation method is electroporation.

25. The method according to one of claims 3 to 24, wherein the first and second nucleic acid molecules are introduced into the host species by co-transformation.

26. The method according to one of claims 3 to 25, wherein the first nucleic acid molecule is introduced into a host cell in which the second nucleic acid molecule is already present.

27. The method according to any one of the preceding claims, wherein the recombination event occurs in vitro.

28. The method of any one of claims 1-27, wherein the recombination event occurs in vivo.

29. Use of a cell capable of expressing a gene encoding a phage annealing protein, or a functional equivalent or fragment thereof, as a host for a cloning method involving repair recombination.

30. Use according to claim 29, with the proviso that if the cell is prokaryotic, the cell does not contain RecE/Redα.

31. Use of a vector system capable of expressing a gene encoding a phage annealing protein, or a functional equivalent or fragment thereof, in a host species for a cloning method involving repair recombination.

32. Use according to claim 31, with the proviso that that if the host species is prokaryotic, the host species does not contain RecE/Redα.

33. A method for altering the sequence of a nucleic acid molecule, said method comprising the steps of:
a) exposing a first nucleic acid molecule to a phage annealing protein, or a functional equivalent or fragment thereof, in the presence of a second nucleic acid molecule, to generate a joint molecule, wherein said first and second nucleic acid molecule share at least two regions of sequence homology;
b) incubating said joint molecule under conditions suitable for repair recombination to occur between said first and second nucleic acid molecules; and
c) selecting a nucleic acid molecule whose sequence has been altered so as to include sequence from said second nucleic acid molecule.

34. A method according to claim 33, with the proviso that the RecE/Redα protein is not present during the course of a sequence alteration reaction that is carried out in a prokaryotic cell.

35. A method for altering the sequence of a nucleic acid molecule, said method comprising the steps of:
a) exposing a first nucleic acid molecule to a phage annealing protein, or a functional equivalent or fragment thereof, to generate a coated nucleic acid molecule;
b) bringing said coated molecule into contact with a second nucleic acid molecule, wherein said first and second nucleic acid molecule share at least two regions of sequence homology; under conditions suitable for repair recombination to occur between said first and second nucleic acid molecules; and
c) selecting a nucleic acid molecule whose sequence has been altered so as to include sequence from said second nucleic acid molecule.

36. A method for cloning a nucleic acid, utilising a method of altering the sequence of a nucleic acid molecule as described in any one of claims 1-28, or 33-35.

37. A method for engineering the sequence of a nucleic acid molecule, comprising the method steps recited in any one of claims 1-28, or 33-35.

38. A method for selection of a desired nucleic acid molecule from a mixture of nucleic acid molecules, said method comprising the steps of:
a) exposing an oligonucleotide molecule that possesses a complementary sequence to the sequence of the desired nucleic acid molecule to a phage annealing protein, or a functional equivalent or fragment thereof, under conditions appropriate for the formation of a coated molecule or a joint molecule complex;
b) incubating the coated molecule or joint molecule complex with the mixture of nucleic acid molecules; and
c) selecting a nucleic acid molecule that is bound to a phage annealing protein.

39. A method according to claim 38, wherein said oligonucleotide includes a tag.

40. A method according to claim 38 or claim 39, wherein said nucleic acid molecule selected in step c) is selected using a method of affinity separation to isolate the tagged oligonucleotide.
